(19) United States
(12) Patent Application Publication
(10) Pub. No.: US 2010/0145399 A1
(43) Pub. Date: Jun. 10, 2010

(54) MULTIFUNCTION DEVICES AND METHODS OF USING THE MULTIFUNCTION DEVICES

(76) Inventors: Pooja Johari, San Diego, CA (US); Satyendra Johari, San Diego, CA (US)

Correspondence Address: LAW OFFICE OF MICHAEL P. EDDY;MICHAEL P. EDDY, 12526 HIGH BLUFF DRIVE, STE. 300, SAN DIEGO, CA, 92130, US

(21) Appl. No.: 12/569,889

(22) Filed: Sep. 29, 2009

Related U.S. Application Data
(60) Provisional application No. 61/194,583, filed on Sep. 29, 2008.

(51) Int. Cl. A61N 1/36; A61N 7/00; A61H 1/00

(52) U.S. Cl. 607/3; 607/59

(57) ABSTRACT

Devices which are used for performing multiple modes of electro, vacuum and ultrasound therapy are disclosed as well as methods of using those devices. One device for providing a constant current and/or a constant voltage to regions of a patient's body includes a housing, at least one microprocessor for generating at least one signal, at least one amplifier circuit for amplifying the one or more signals, at least one feedback circuit for delivering at least one constant current to a patient's body, at least one lead wire socket located on the exterior of the housing, at least one pair of electrodes connected to the one or more lead wire sockets, wherein the electrodes are placed in communication with at least one region of the patient's body so that an output current and or voltage is delivered to one or more of the skin regions of the patient's body. Additionally, a controller is used to activate the constant current and/or voltage and it is also used to select one or more selected modes for the constant current and/or voltage.

### CROSS-REFERENCES TO RELATED PATENT APPLICATIONS

This application claims the benefit of U.S. provisional application Ser. No. 61/194,583, filed Sep. 29, 2009 which is incorporated herein by reference in its entirety for all purposes.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISK APPENDIX

Not Applicable.

### BACKGROUND

### A. Field

Embodiments of the claimed subject matter relate to multifunctional medical devices and methods for using the multifunctional devices, and more particularly, to devices for providing electro therapy, vacuum or ultra sound therapy to areas of a patient's body including but not limited to providing electric stimulation to the cranial region of a patient.

### SUMMARY

The claimed subject matter relates to medical devices for providing a constant current and/or a constant voltage to regions of a patient's body. Embodiments include devices and methods of using those devices for performing multiple modes of electro, vacuum and ultrasound therapy.

The modes include an Interferential (IF) mode, an EMS Mode, a Pre-modulated IF mode, a Micro Current mode, a Galvanic-DC mode, a CES Mode, an Ultrasound Therapy mode, and a Vacuum therapy mode and additional protocols can be used with combining or varying one or more of the aforementioned modes.

Embodiments of the device include a housing, at least one microprocessor for generating at least one signal, at least one amplifier circuit for amplifying the one or more signals, at least one feedback circuit for delivering constant current to a patient's body, at least one lead wire socket located on the exterior of the housing, at least one pair of electrodes connected to the one or more lead wire sockets, wherein the electrodes are placed in communication with at least one region of the patient's body so that an output current and or voltage is delivered to one or more of the skin regions of the patient's body. Additionally, a controller is used to control the constant current and/or voltage and it is also used to select one or more selected modes for the constant current and/or voltage.

In several of the embodiments, the selected mode can be one or more of the following modes: an interferential mode, an electric muscle stimulation mode, a pre-modulated interferential mode, a galvanic-DC mode, a micro current mode, a cranial electric stimulation mode, an ultrasound therapy mode, and a vacuum therapy mode.

Other embodiment also include a display located on the exterior of the housing of the device for providing audio and/or visual information to said user. In some of these embodiments, the display is a touch screen LCD.

In several of the embodiments, the output current can be valued at approximately 100 mA for the interferential mode, the electric muscle stimulation mode and/or the pre-modulated interferential mode. In several embodiments, the output current can be valued at approximately 30 mA in the galvanic-DC mode, 400 μA in the micro current mode, and 1.5 mA in cranial electric stimulation mode but these values may be adjusted according to the desires of the user and/or patient.

### BRIEF DESCRIPTION OF THE DRAWINGS

The accompanying drawings, which are incorporated in and constitute a part of this specification, illustrate embodiments of the claimed subject matter, and, together with the description, further explain the claimed subject matter.

In the drawings,

FIG. 1 is a front view of a multifunction device in accordance with embodiments of the claimed subject matter;

FIG. 2 is a block diagram illustrating various components and their interface in accordance with embodiments of the claimed subject matter;

FIG. 3 (a) to FIG. 3 (i) are electrical schematics of a main controller board which covers an interface section in accordance with embodiments of the claimed subject matter;

FIG. 4 (a) to FIG. 4 (g) are electrical schematics of a estim board used with embodiments of the claimed subject matter;

FIG. 5 (a) to FIG. 5 (c) are electrical schematics of an ultrasound board used with embodiments of the claimed subject matter;

FIG. 6 is a block diagram of an ultrasound applicator board used with embodiments of the claimed subject matter;

FIG. 7 is an electrical schematic for an encoder board used with embodiments of the claimed subject matter;

FIG. 8 (a) illustrates an exemplary waveform with resistive loads for IF therapy in accordance with embodiments of the claimed subject matter;

FIGS. 8 (b) to 8 (k) illustrates exemplary waveform with resistive loads for EMS therapy in accordance with embodiments of the claimed subject matter;

FIG. 8 (l) illustrates an exemplary waveform with resistive loads for pre-modulated interferential therapy in accordance with embodiments of the claimed subject matter;

FIG. 8 (m) illustrates an exemplary waveform with resistive loads for Micro current therapy in accordance with embodiments of the claimed subject matter;

FIG. 8 (n) illustrates an exemplary waveform with resistive loads for Galvanic-DC therapy in accordance with embodiments of the claimed subject matter;

FIG. 8 (o) illustrates an exemplary waveform with resistive loads for CES therapy in accordance with embodiments of the claimed subject matter; and

FIG. 9 is a flow diagram illustrating exemplary logic performed by a microcontroller used with the embodiments of FIG. 1.

### DETAILED DESCRIPTION OF THE EMBODIMENTS

Embodiments of the claimed subject matter are directed to devices and methods for performing electro, vacuum and ultra sound therapy. In describing the disclosed subject matter, including those embodiments illustrated in the drawings, specific terminology is employed for the sake of clarity. Although these parameters will now be discussed in further detail, these descriptions are not an exhaustive explanation of all possible variations in structure and operation. It will be apparent to those skilled in the art that various other changes or modifications can be made without departing from the spirit and scope of the embodiments presented herein. It should be further apparent that any or all combinations of the individual described variations with the disclosed embodiments are possible.

The presently described embodiments use software and hardware or embedded software in hardware (such as a micro controller programmed with software) to generate and control output signals. In an exemplary embodiment, the multi-function device according to the present invention has 8 channels and the following modes: (1) Interferential (IF) mode; (2) EMS Mode; (3) Pre-modulated IF mode; (4) Micro Current mode; (5) Galvanic-DC mode; (6) CES Mode; (7) Ultrasound Therapy mode; and (8) Vacuum therapy mode.

In one embodiment, an IF stimulation with up to three outputs viz. output 1, 2, 3 (six channels/12 electrodes) is used. Other embodiments may use more or less outputs with any one or more of a number of modes. The described embodiments can be used to treat three body areas simultaneously with the IF stimulation (for example, with the use of 4 electrodes for each body area). In another embodiment used for EMS stimulation, two outputs viz. output 1, 2 (four channels/8 electrodes) are used. Many of the embodiments can be used to treat four body areas (abdominal/buttocks) simultaneously with the EMS stimulation. Other embodiments may be used for massage therapy with EMS in which one body area is covered.

In Pre-modulated IF stimulation two outputs viz. output 1, 2 (four channels/8 electrodes) are used. Thus, the device according to the present invention can be used to treat four body areas simultaneously with the Pre-modulated IF stimulation. The micro-current stimulation is given through output 4 (one channel/ two conductive rollers). In Galvanic-DC mode stimulation maximum one output out of the outputs 1, 2, or 3 (two channels/4 electrodes) can be used. Thus, the device according to the present invention can be used to treat two body areas simultaneously with the Galvanic-DC stimulation (2 electrodes for each body area). The CES stimulation is given through output 4 (one channel/two ear clips). Ultrasound therapy can be given through a separate ultrasound socket with the help of an ultrasound applicator. Vacuum therapy can be given through a separate Vacuum socket with the help of vacuum cups. Thus, any of the Interferential (IF) mode, EMS Mode, Pre-modulated IF mode, Micro Current mode, Galvanic-DC mode or CES Mode can be selected concurrently depending upon the availability of the free channels viz. 1, 2, 3 and 4. Vacuum or ultrasound therapy can be started any time irrespective of any of the other running stimulation/therapy.

The multifunction device according to embodiments of the claimed subject matter may also provide at least the following benefits: a single device for providing electrotherapy for both mind as well as body, the provision of both vacuum and ultrasound therapies, and the ability to provide a constant current to different body areas. In several embodiments, the selected current does not need to be changed with the altered resistance of different body areas and the device may continue to produce the same current that was selected by the user.

Embodiments can be more reliable and accurate since no calibration is required in the device. These features also help to lower production costs and can also help make the described embodiments more user friendly. Further, in the described embodiments, an external oscillator is not required for the generation of various waveforms and this also helps make the device more cost effective. In addition, hazards due to device failure such as over heating and the like may be avoided due to the use of an ADC feedback circuit.

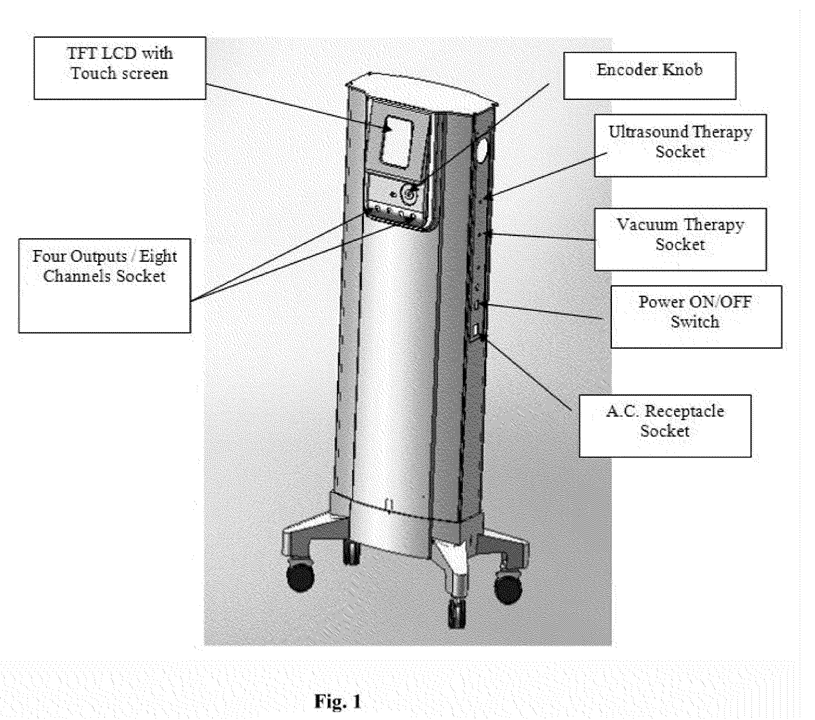

In the drawings, FIG. 1 is the front view of the multi-function device according to embodiments of the claimed subject matter. The device uses software (micro controller) to generate and control output signals. In exemplary embodiments, the present invention is an eight-channel IF/EMS/Pre-modulated Interferential/Micro current/Galvanic-DC/CES stimulator and Ultrasound and vacuum therapy device.

In exemplary embodiments the device has selectable modes of stimulation/therapy with parameters that can be individually controlled using a touch screen LCD control panel and display which is located on the exterior of the device housing. The LCD screen contains keys/tabs for the selection of mode in the particular selected channel and for the treatment time selections. The screen displays the intensity level of the stimulation/therapy. In exemplary embodiments, touch screen LCD control panel have START and STOP keys, time increase and decrease keys, PAUSE and RESUME keys and output channel and mode selection keys. Encoder knob for the intensity control, AC supply socket and power ON/OFF switch are provided on the exterior of the device housing. Ultrasound and vacuum output sockets are also provided on the exterior of the device housing. For easy movement of the device wheels are provided on the bottom of the device.

The On/Off button is depressed in order to turn on the device. When the device is turned on, the LCD glows and the user can select the desired mode(s) by touching the respective key/tab on the screen. Once the mode is selected the user can start the stimulation/therapy by touching the START key. The user can increase or decrease the intensity by using the encoder knob and the selected intensity level is displayed on the screen. The PAUSE key stops stimulation/therapy but leaves the device 10 on. Pressing the PAUSE key again resumes the stimulation/therapy at the same point in the stimulation/therapy program. The user can stop the stimulation/therapy by touching the STOP key.

Exemplary embodiments include four lead wire sockets for electric stimulation and one socket each for ultrasound and vacuum therapy. A four pole electrode lead wire is plugged into the lead wire socket. Six pairs (twelve) of electrodes are provided for IF/EMS/Pre-modulated Interferential and Galvanic-DC stimulation. One pair of conductive gloves is provided for Micro current stimulation. One pair of ear clip electrode is used for CES stimulation. Ultrasound is applied through Ultrasound applicator and vacuum cups are used to give the vacuum therapy. Ball electrodes are used for giving the galvanic-DC stimulation.

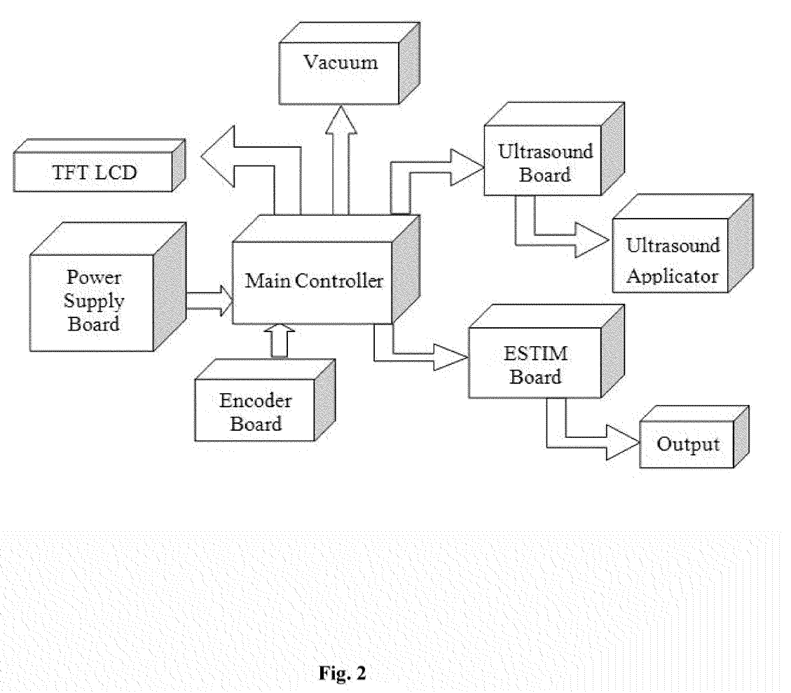

FIG. 2 is an exemplary block diagram illustrating various components and their interface for forming a device in accordance with the present invention. Physically, system will consist of following components Main Controller Board, ESTIM eight channel board, Ultrasound Board, Ultrasound Applicator Board, TFT LCD with Touch Screen, Encoder Board, and Power Supply Board.

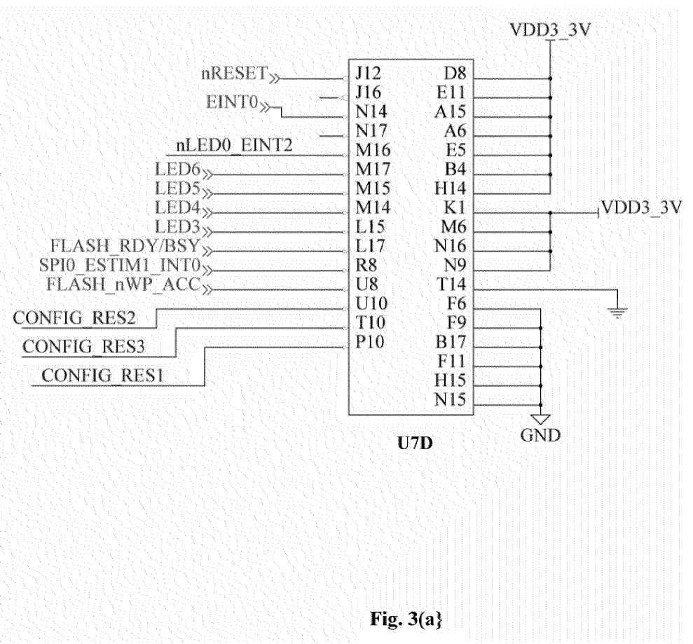

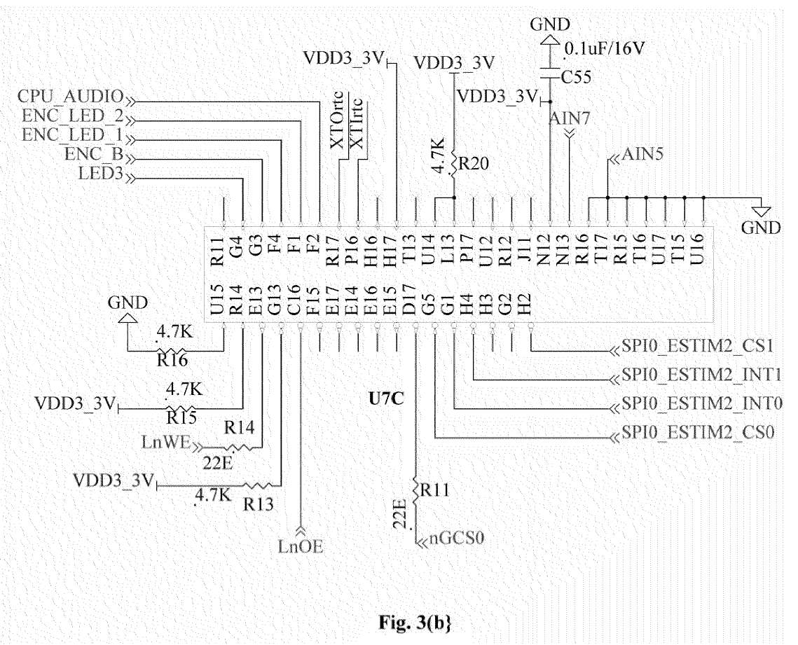

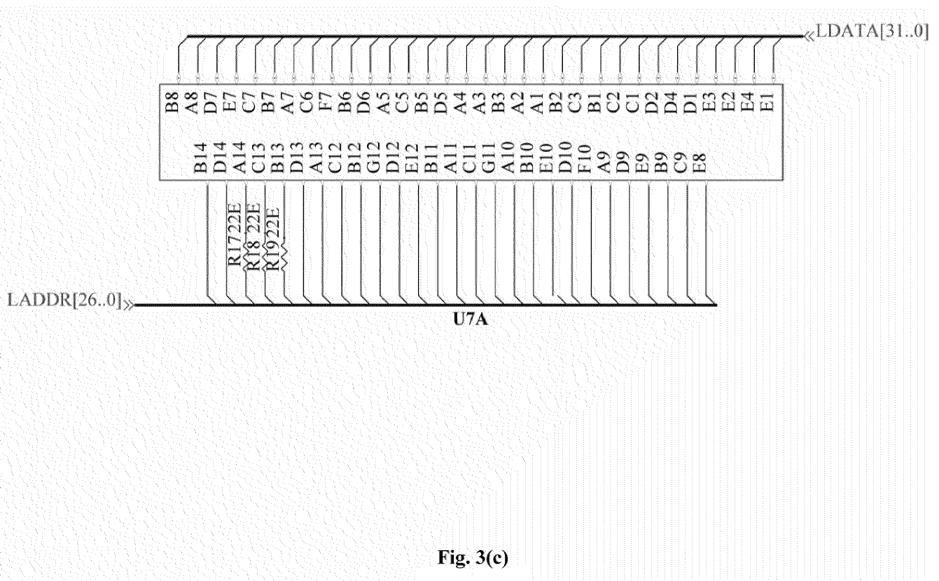

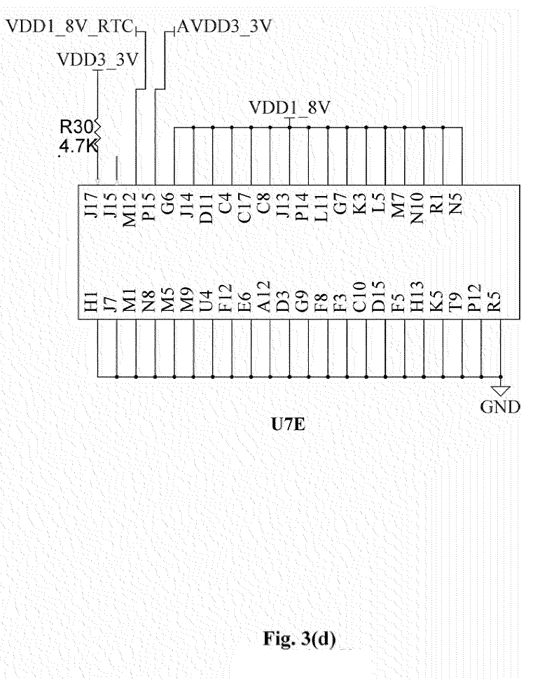

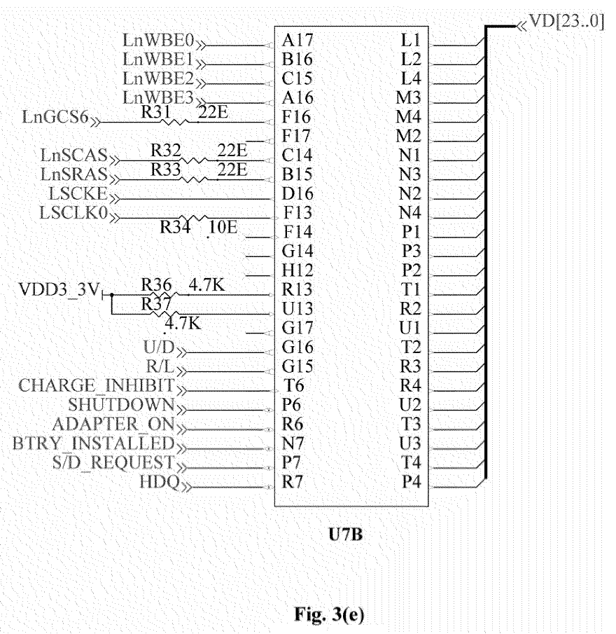

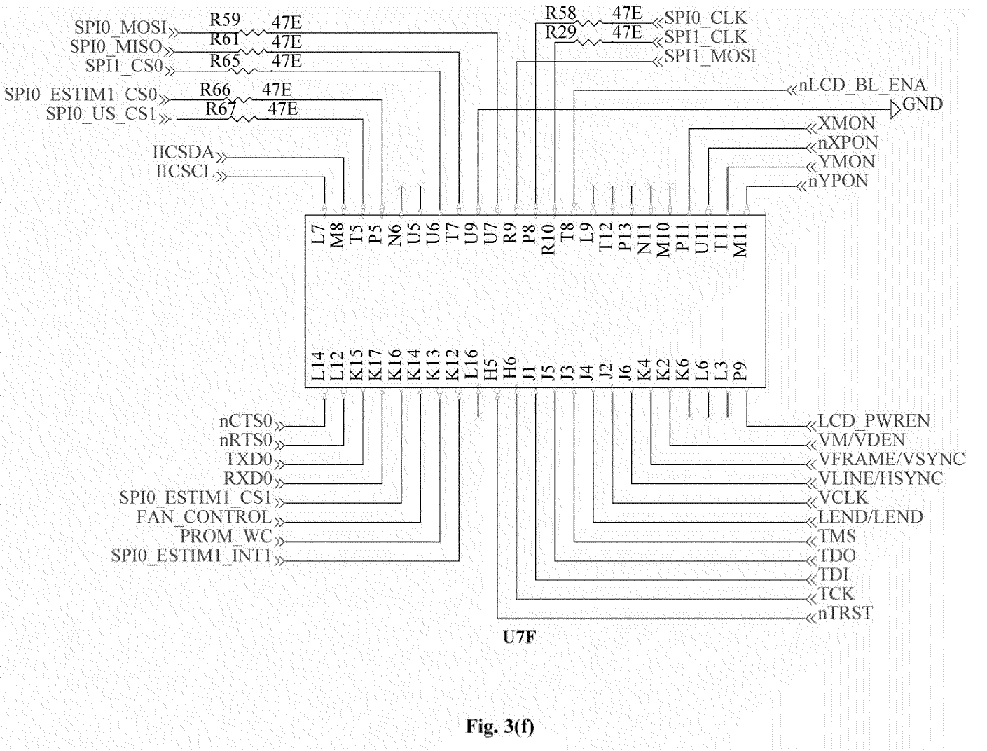

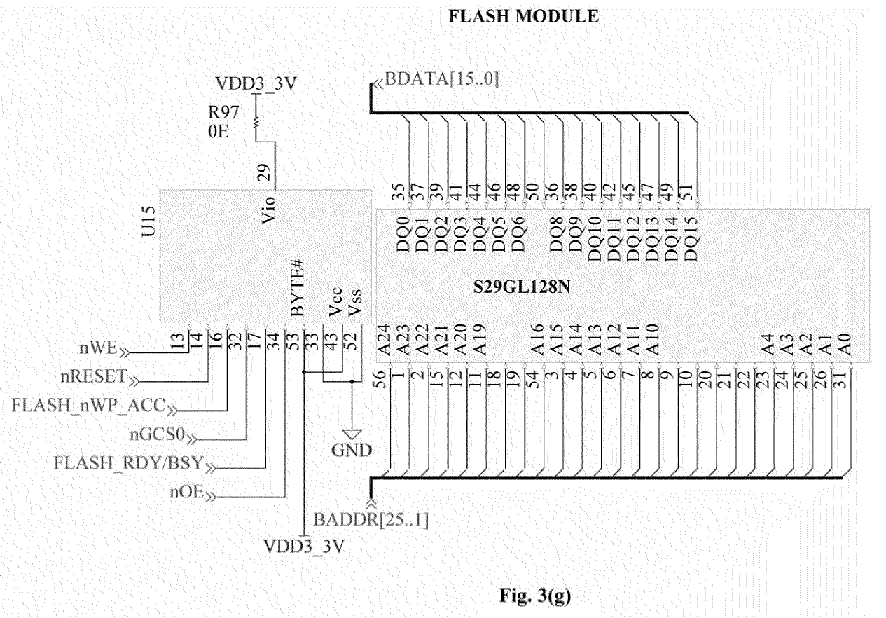

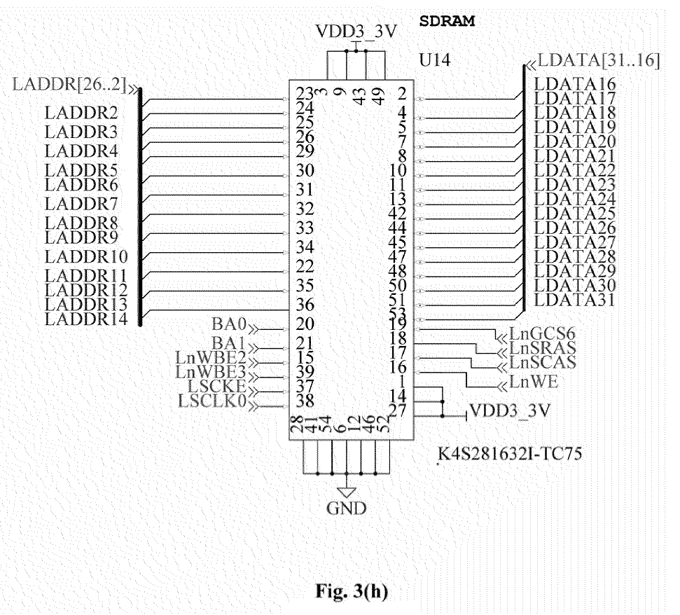

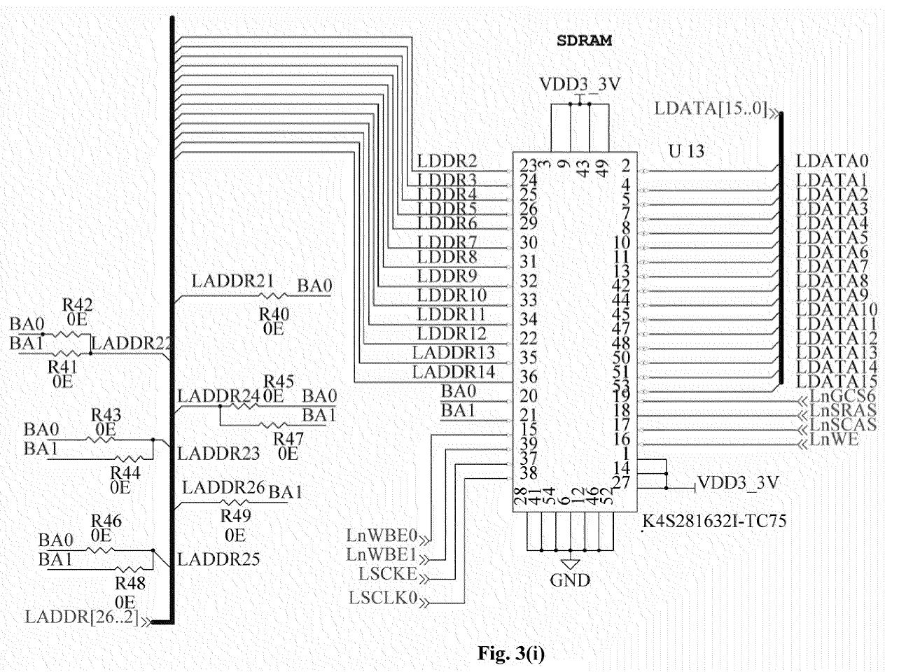

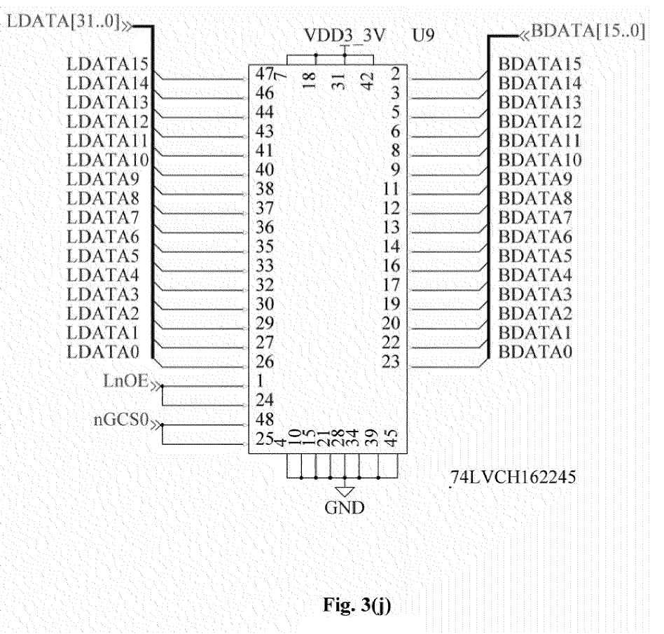

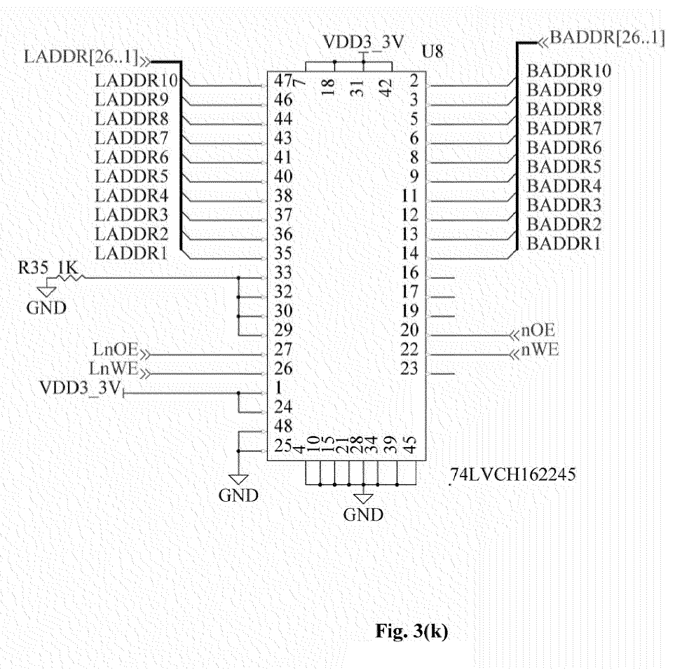

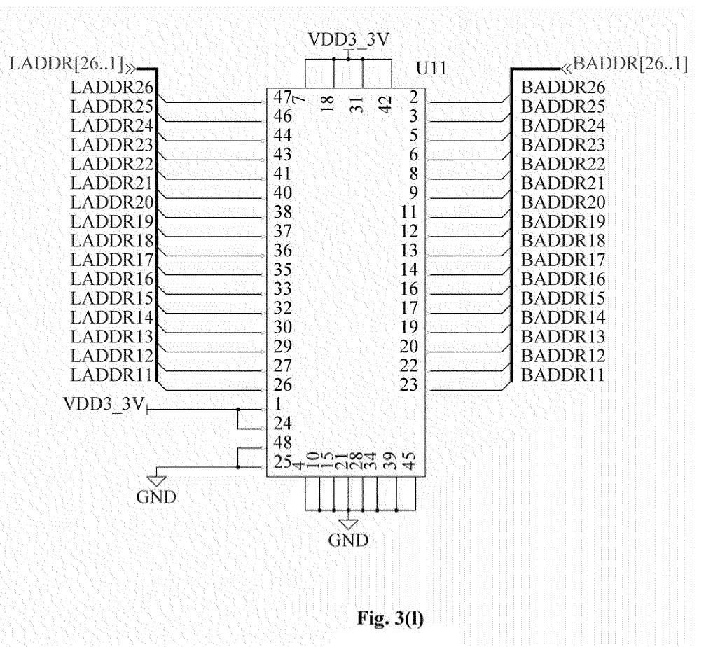

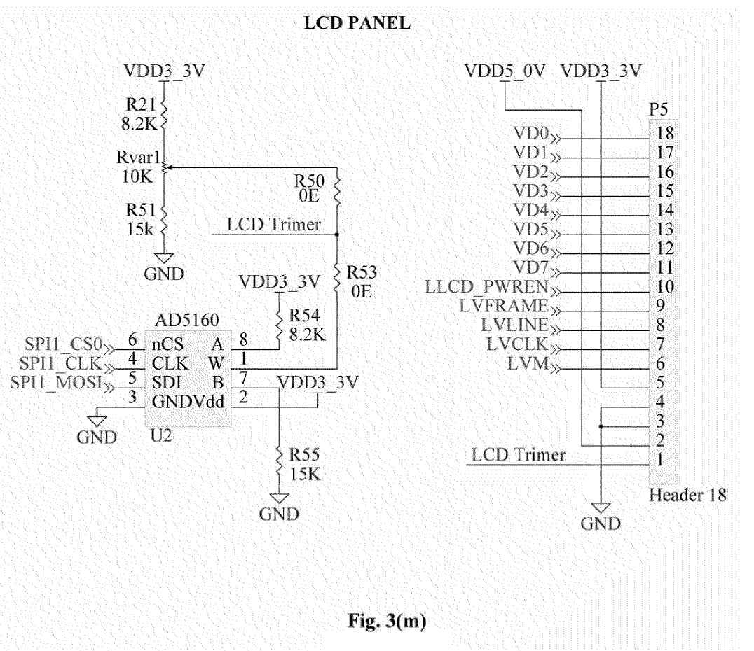

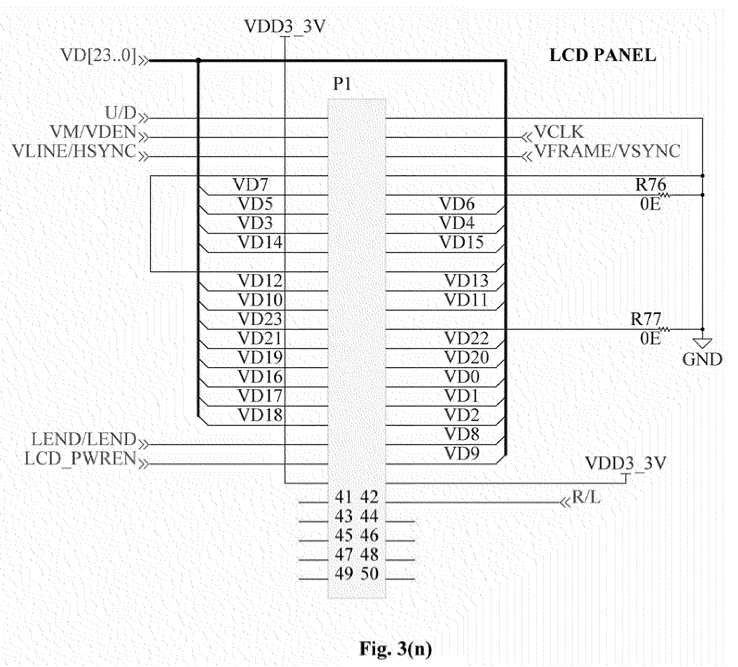

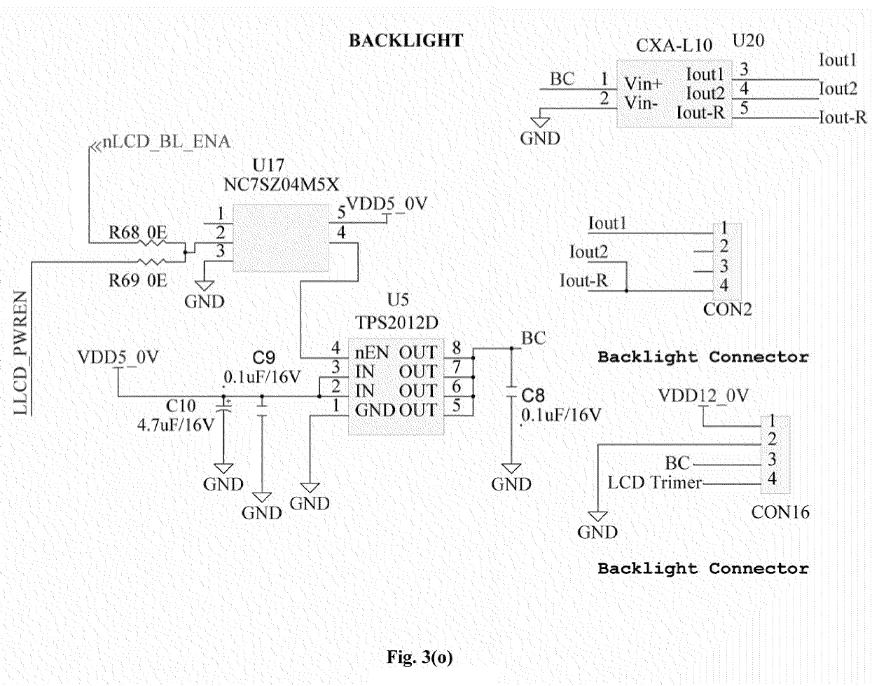

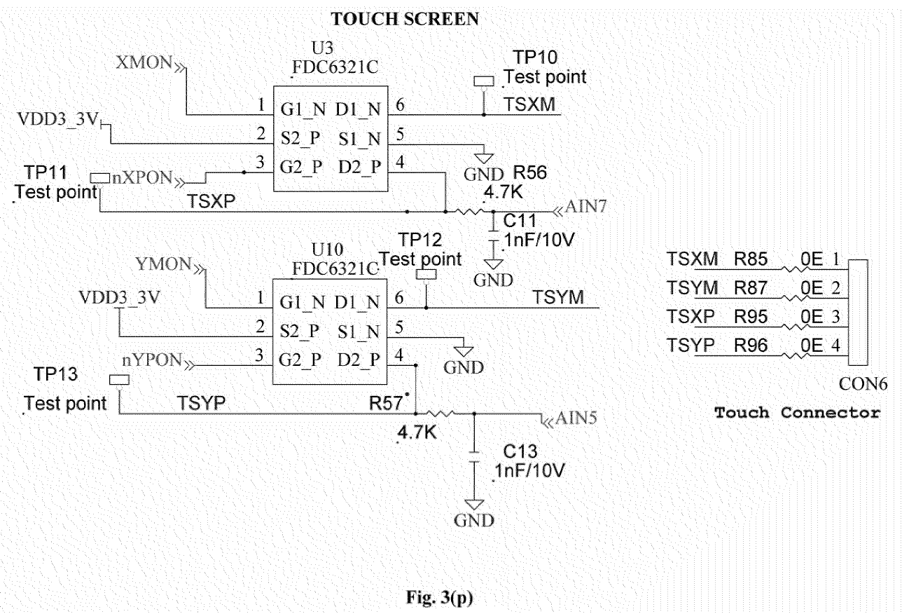

FIG. 3 (a) to FIG. 3 (i) are electric schematics for a Main controller board and TFT LCD. Main controller board contains microcontroller for interfacing. It has 8 MB flash memory and 128 MB SDRAM. The microcontroller is capable of running Windows CE and UNIX operating system. Preferably Windows CE 5.0 operating system is used. DOTNET compact framework 2.0 based application is used to control the Electric wave generator (ESTIM) and Ultrasound Boards and vacuum motor.

TFT LCD with Touch Screen is used as a user control and display. Software which is used as control software for ESTIM board, Ultrasound board, Vacuum takes commands from LCD directly. User can interface the LCD touch screen to the controller. Touch screen is based on the voltage formation. If the screen is touched, it will generate the voltage according to the figure position and that voltage is supplied to the controller A2D section. Now A2D section calculates the position of figure according to the voltage level.

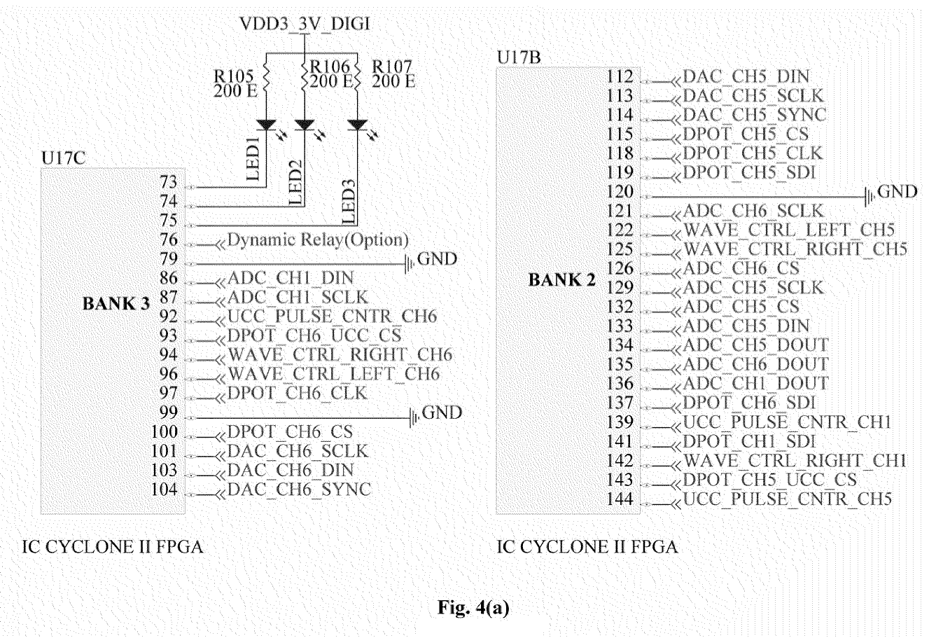

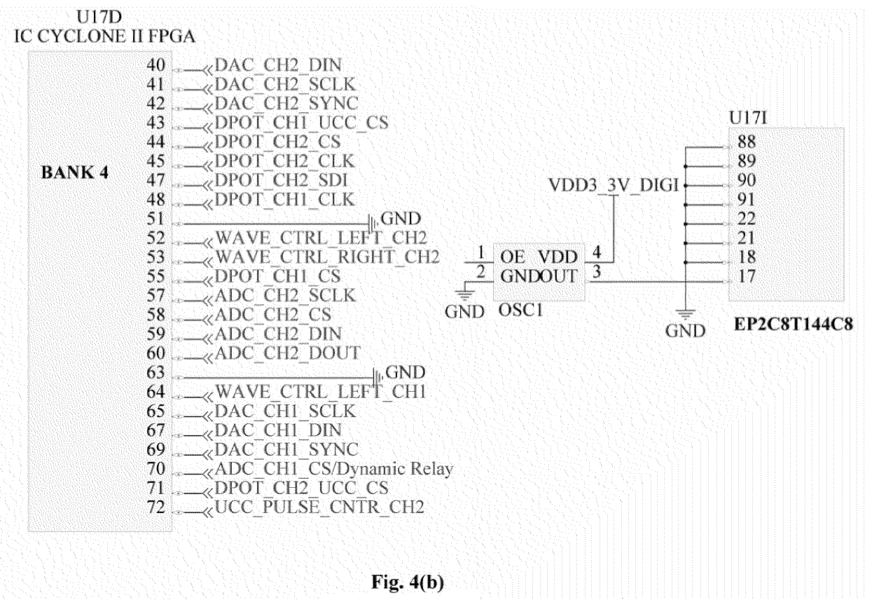

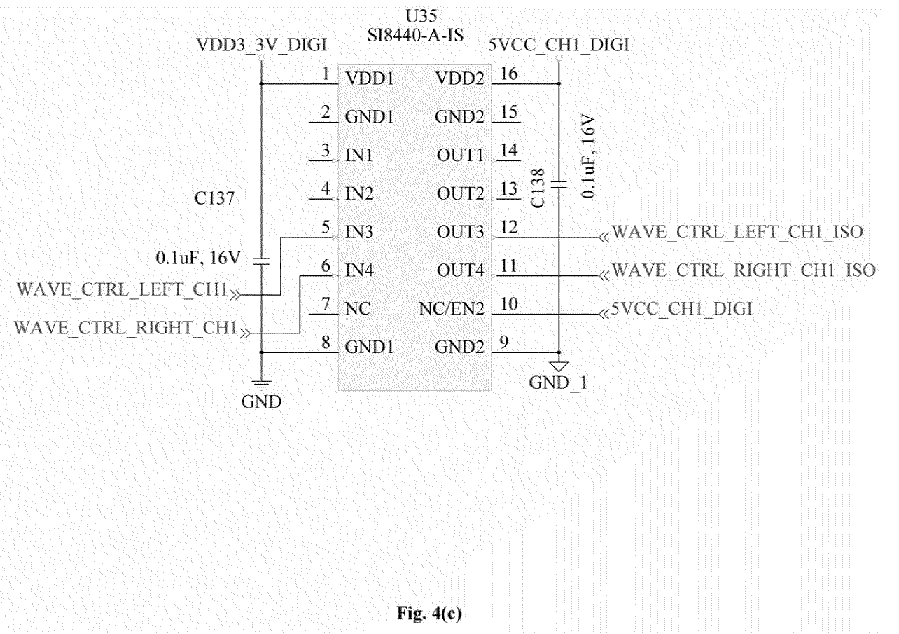

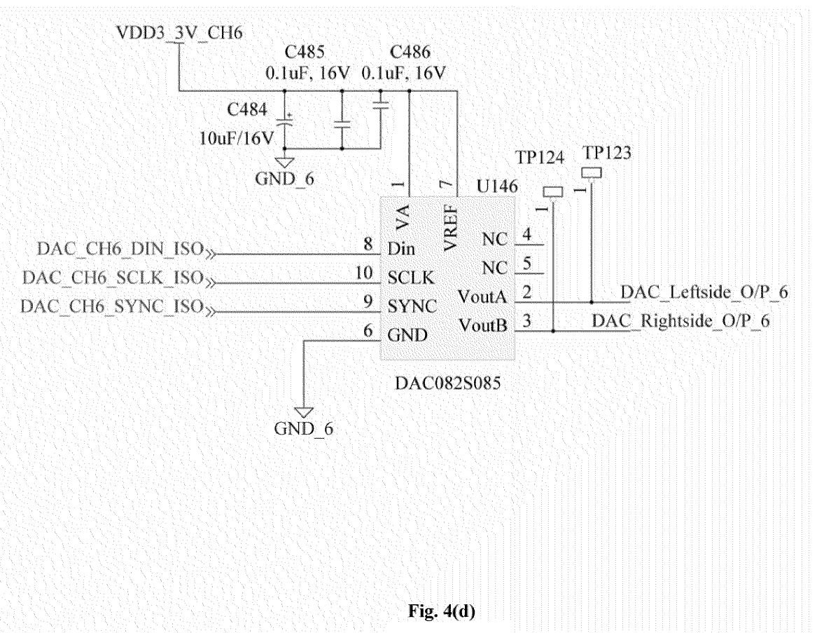

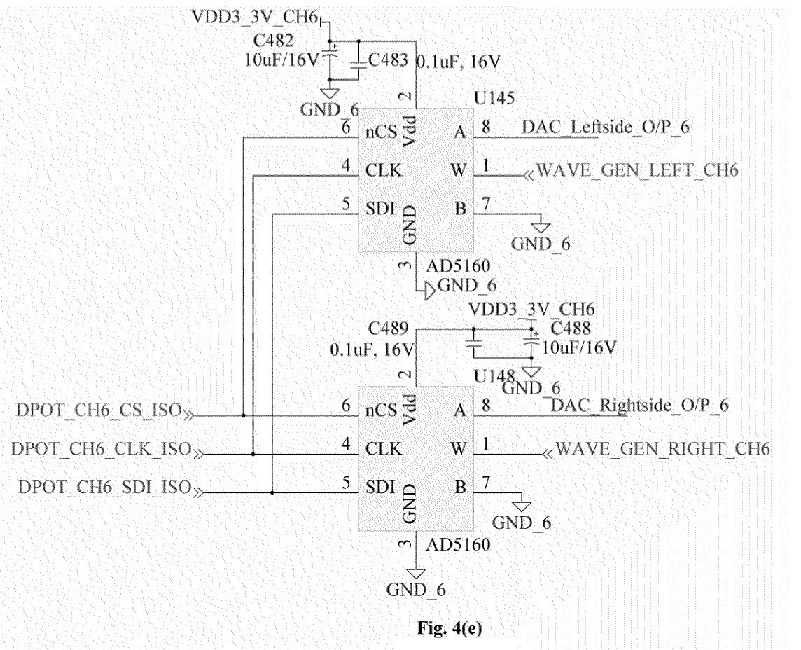

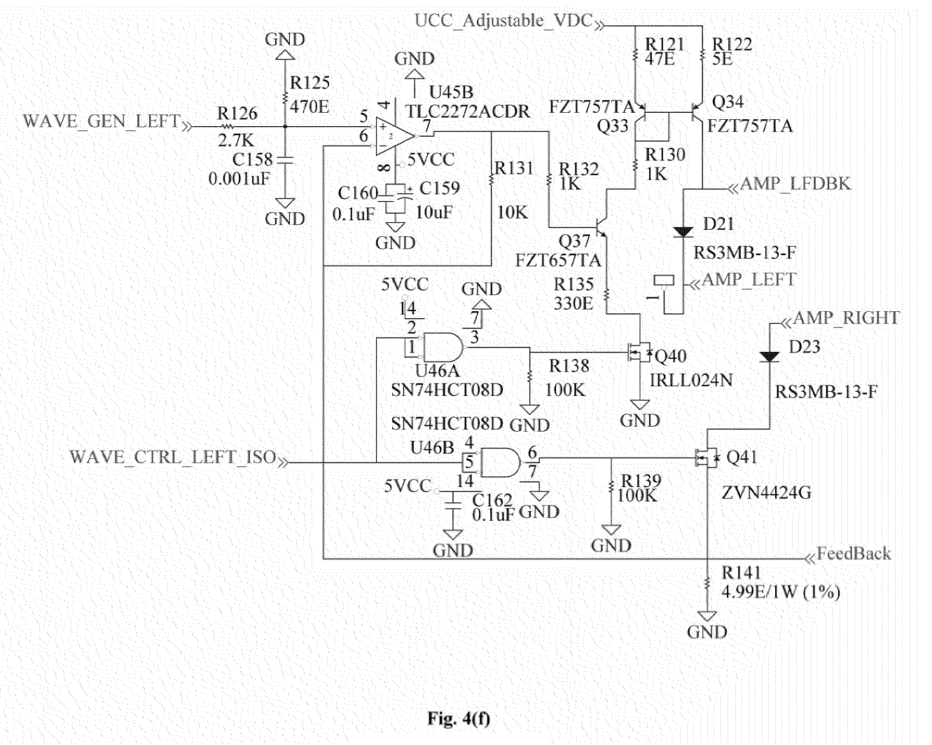

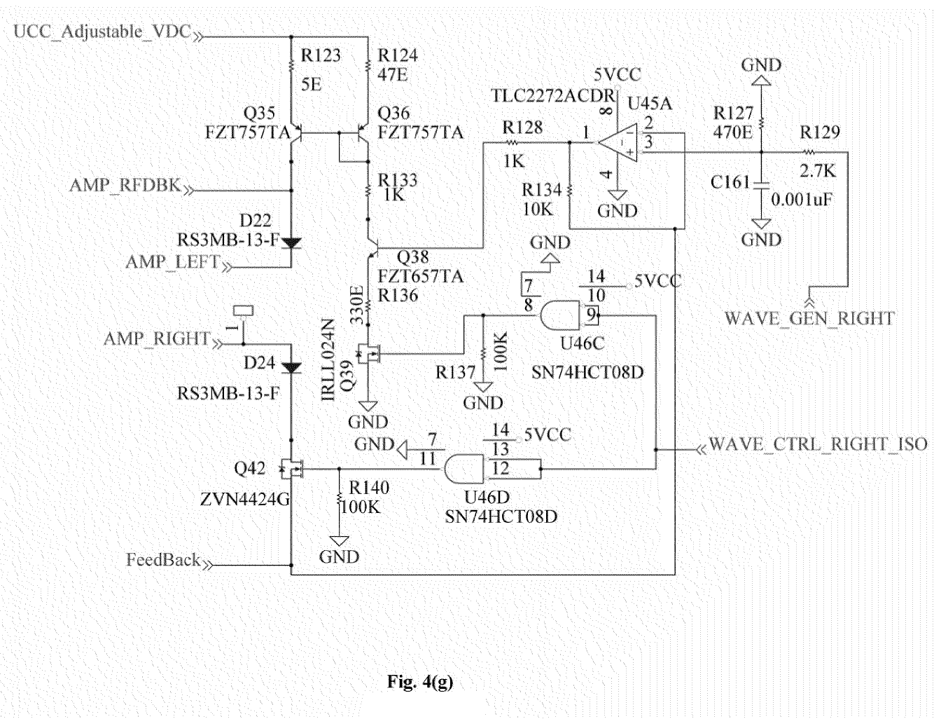

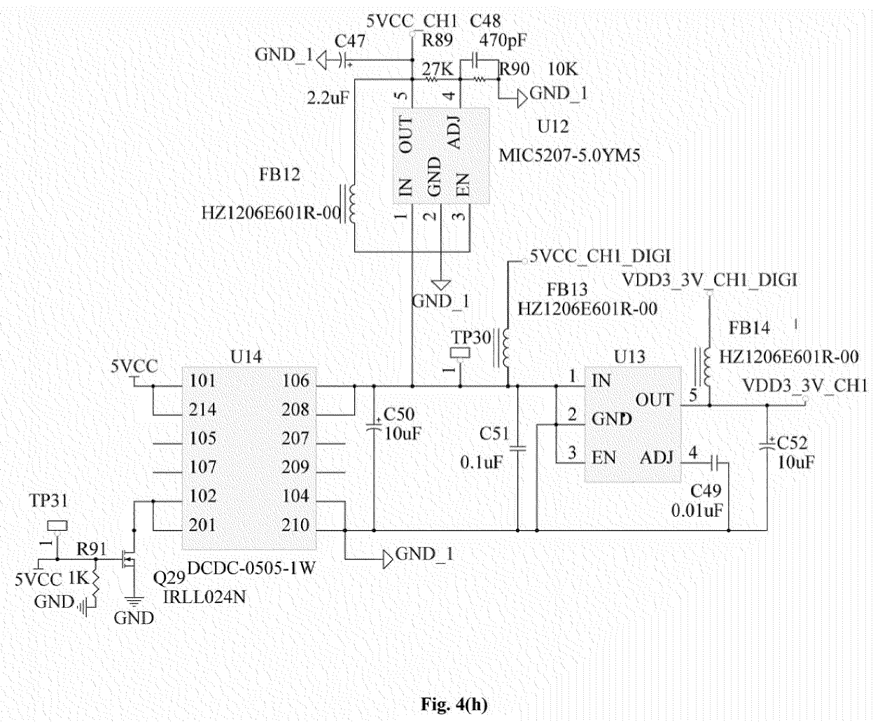

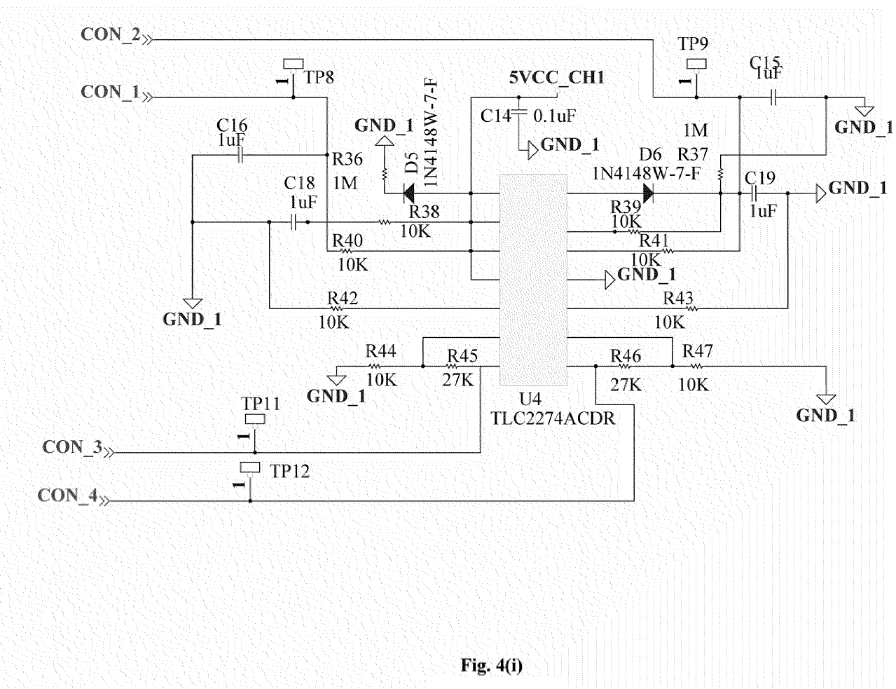

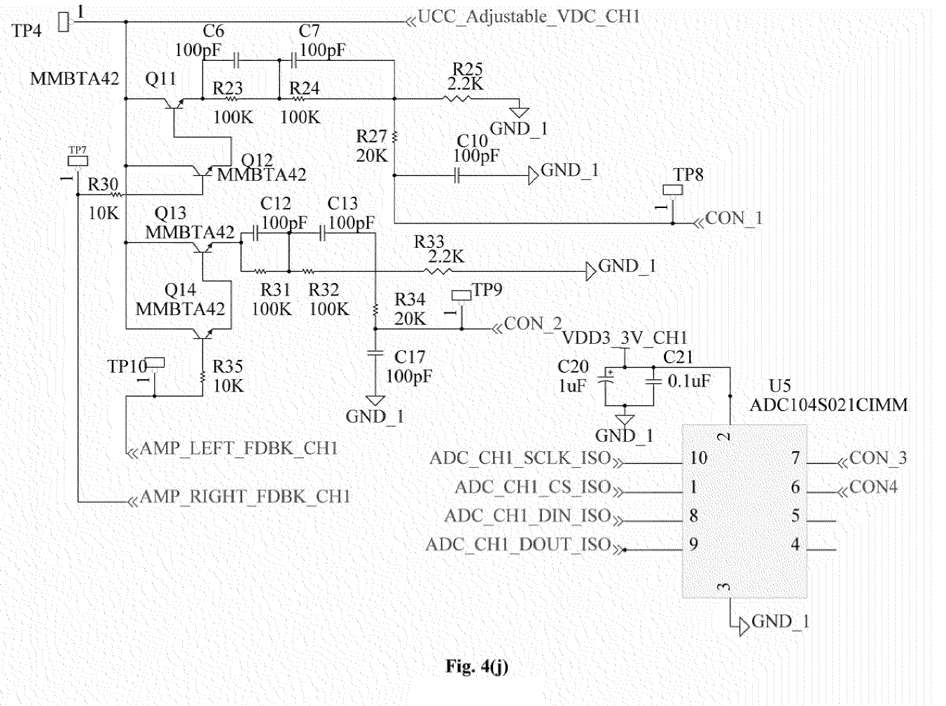

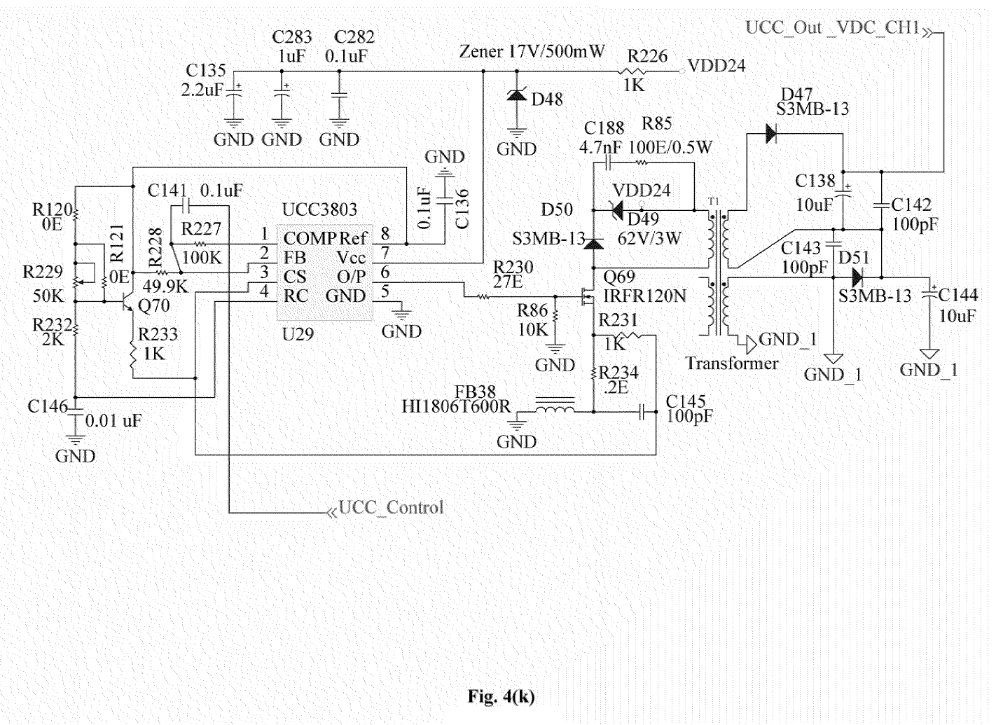

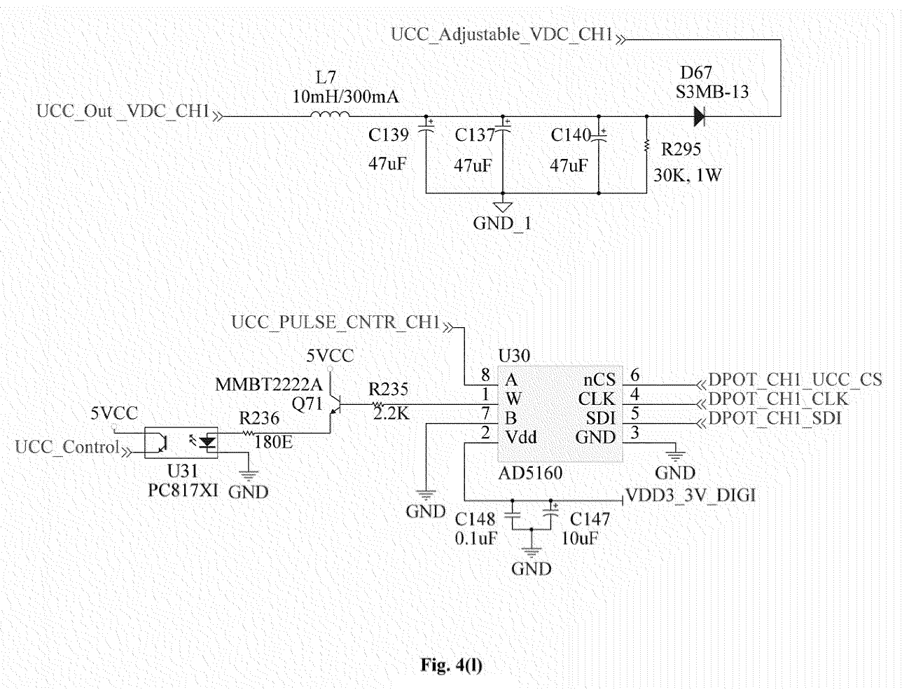

FIGS. 4 (a) to 4 (g) is of the two Altera Cyclone II FPGA in ESTIM board. This board is responsible for generating different type of electrical signal. This board has eight output channels; on each channel different types of waveforms can be generated. In ESTIM board Digital To Analog Converter (DAC) is used to get analog signals. The analog signals are controlled through digital pot. These controlled analog signals are amplified through the amplifier circuit. The required DC supply for the amplifier is provided by IC UCC—3803 circuit. The whole output is controlled through Analog To Digital (ADC) feedback circuit.

In ESTIM board, amplifier consists with various components. It is working on constant current principle. Output current is controlled through output load feedback. The device has been designed according to the capability to drive enough current to human body through various kinds of electrodes. All eight channels amplifier are isolated with each other.

ADC feedback circuit is designed to control various hazard failures. This circuit gives information about component failure, proper electrode connection, overload information, and it also controls the required current.

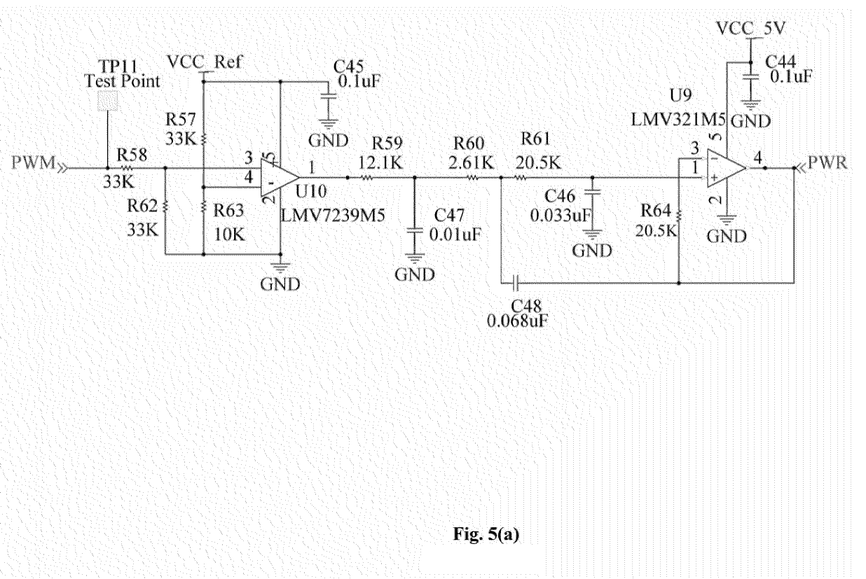

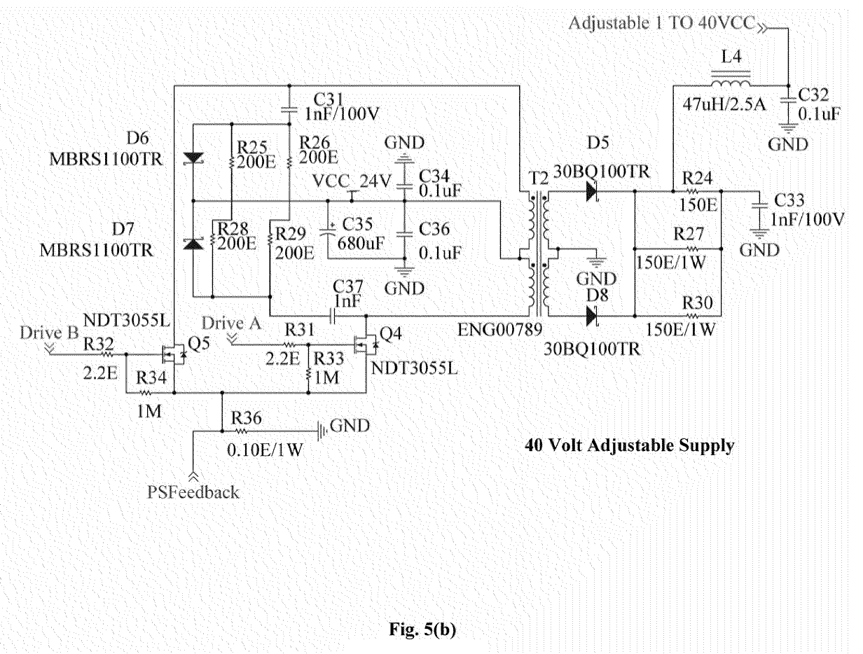

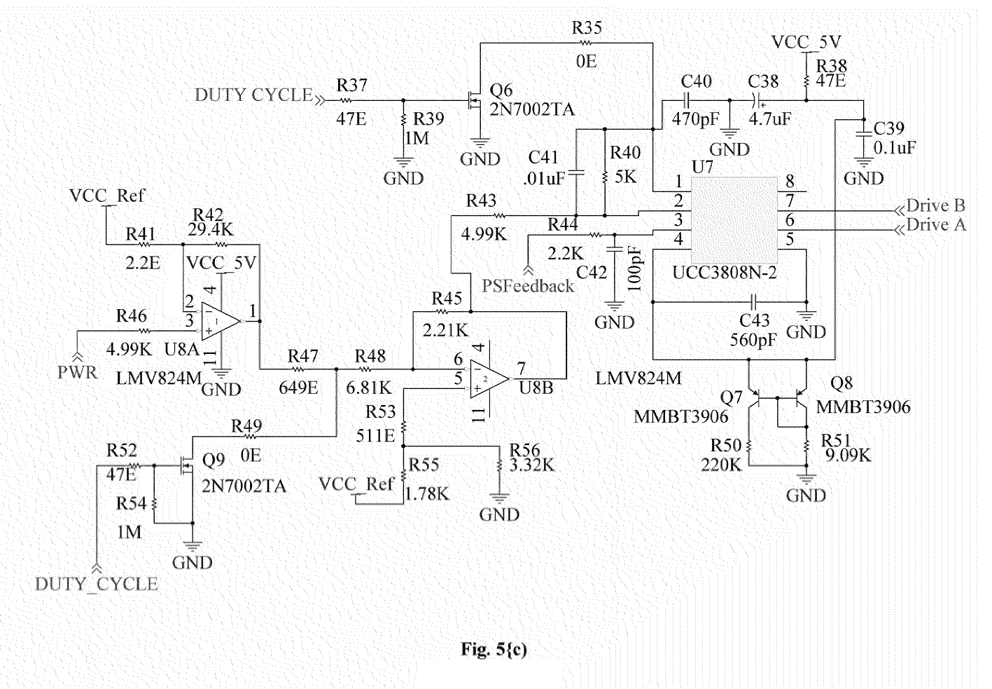

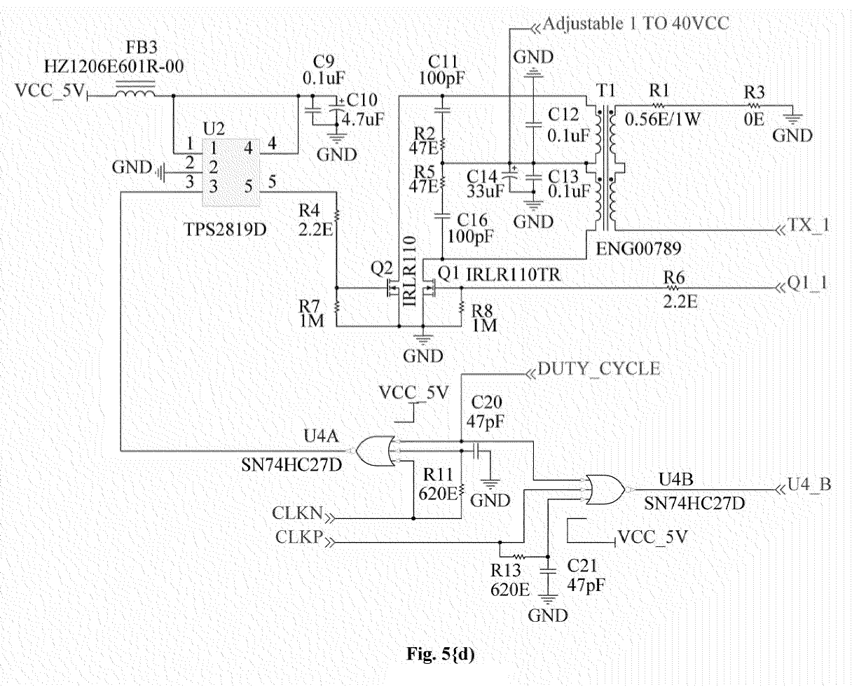

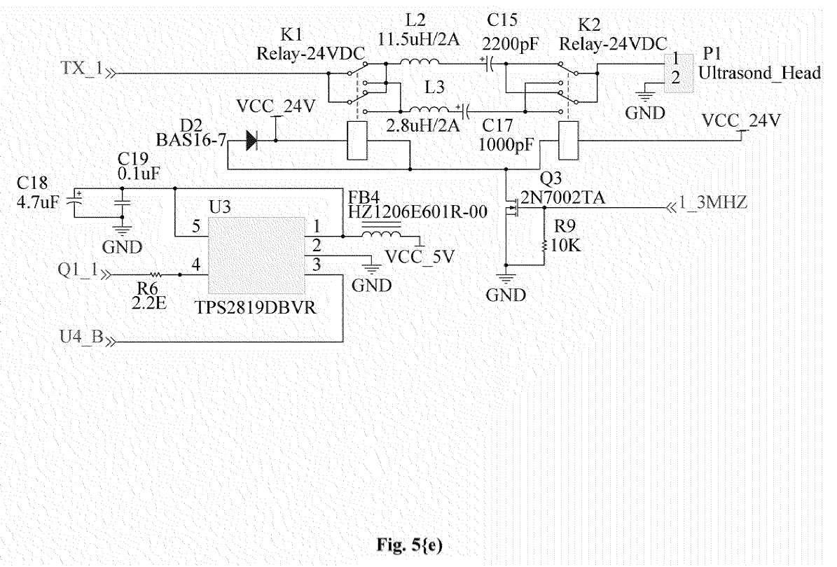

FIG. 5 is a diagram of the Ultrasound Board. It generates signals of 1 MHz and 50% duty cycle with 3 W/cm2 intensity, a feedback circuit in ultrasound board maintains the output power constant by sensing the flow of current in the circuit, the main purpose of this section is to deliver the maximum output at low power. This is done by turning on only single side of output at a time. LC (inductor capacitor) filter circuit is for 1 MHz. This section is used to select appropriate LC filter circuit according to the frequency. PLL (Phase Locked Loop) is used to generate the 1 MHz signal. PLL is directly controlled by the microcontroller using I2C communication. Ultrasound Applicator board contains with micro controller, which communicates to ultrasound board through I2C line and accepts commands to run ultrasonic transducer connected to it. Thermal sensor is used to sense the temperature of output head.

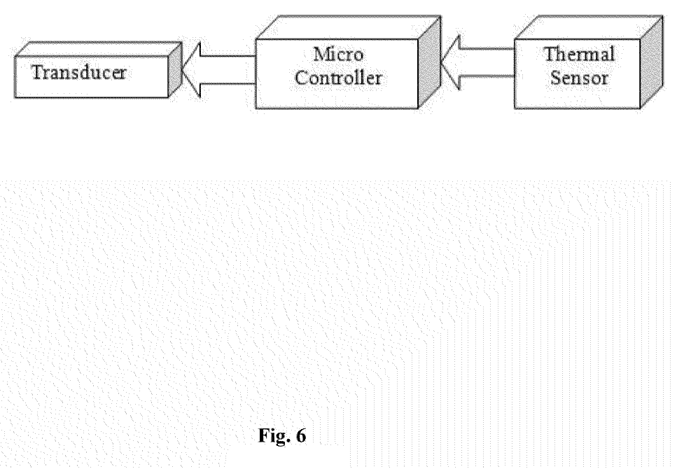

FIG. 6 is operation block Diagram for Ultrasound Applicator board of a multifunction device formed in accordance with the present invention.

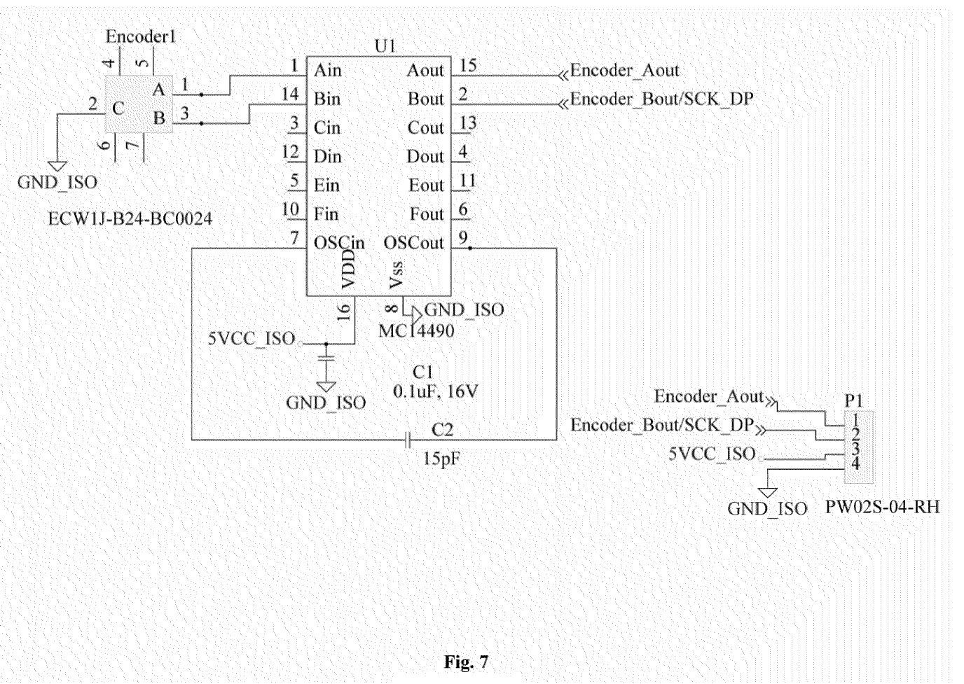

FIG. 7 is a diagram of the Encoder board consisting of an analog knob for intensity increment and decrement. Device also requires 24 VDC/5 Amp power supply for the 24 VDC/5 amp SMPS for universal input AC. In according to the present invention output current will be constant, based on a variable load (1.5 Kohm). Preferably, in defining a mode, a user can specify whether the current is constant or based on a variable load. In device for selected mode output can be adjusted according to requirement, in constant current topology, output current will be same for all patients.

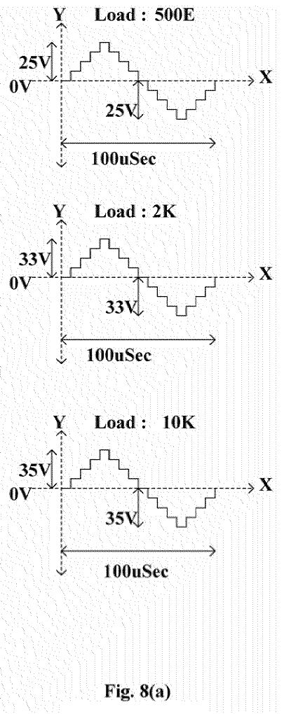

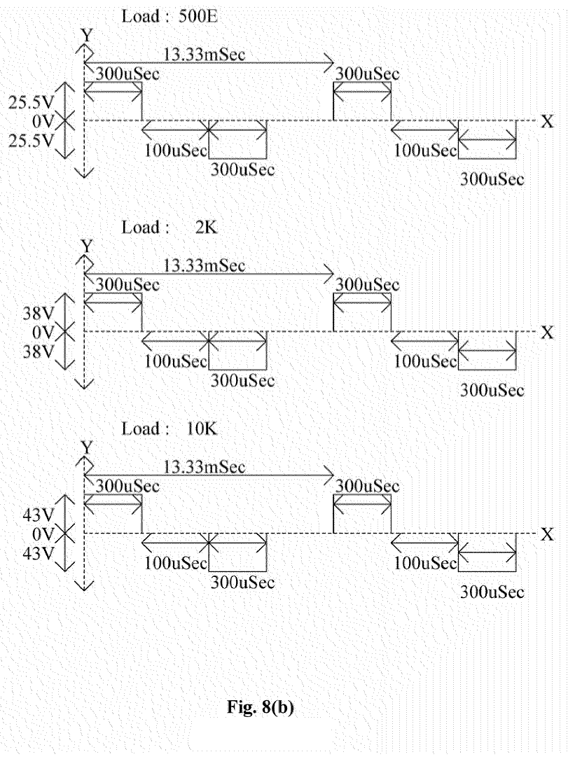

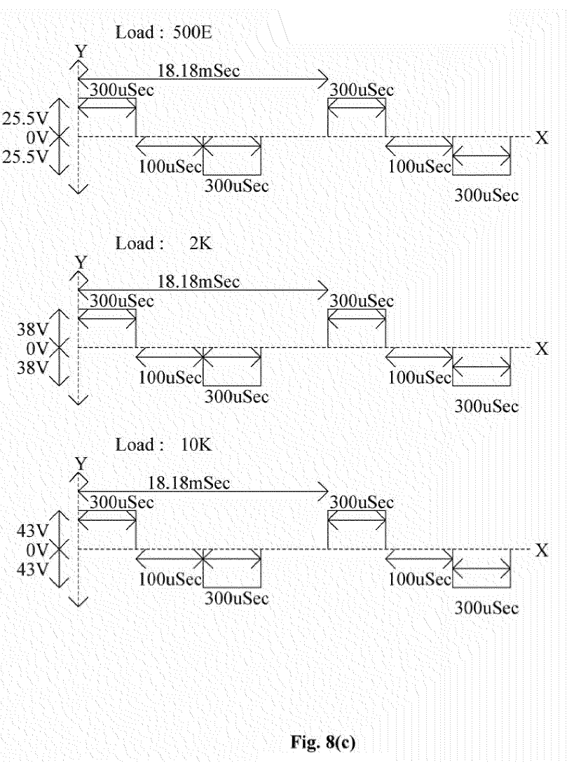

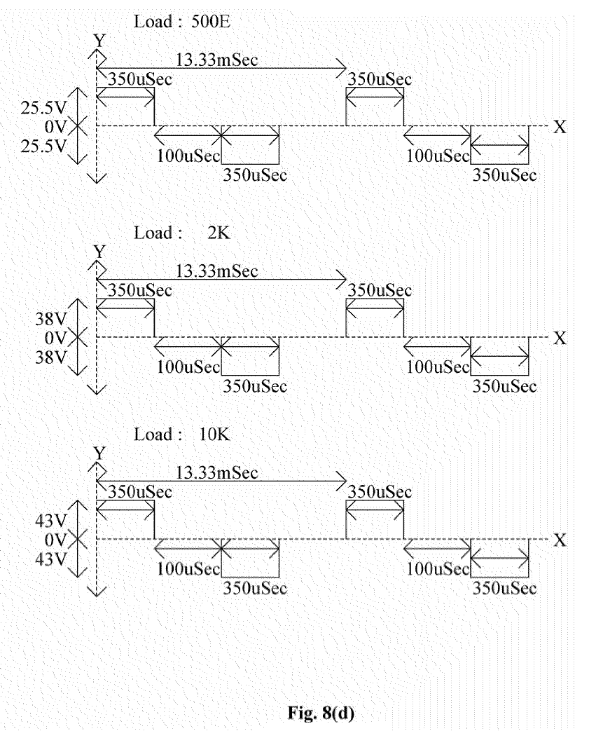

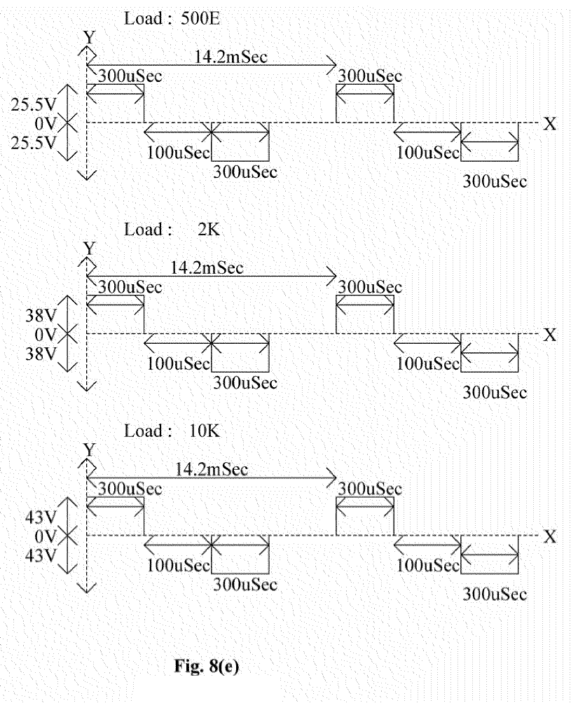

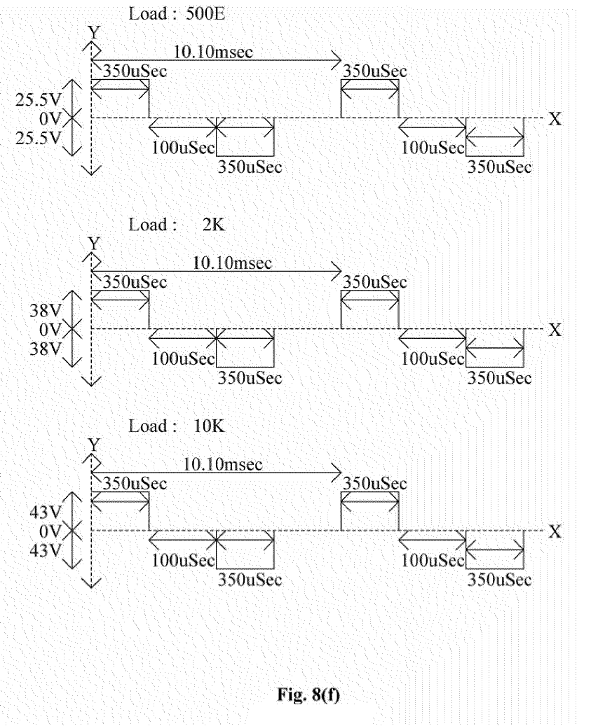

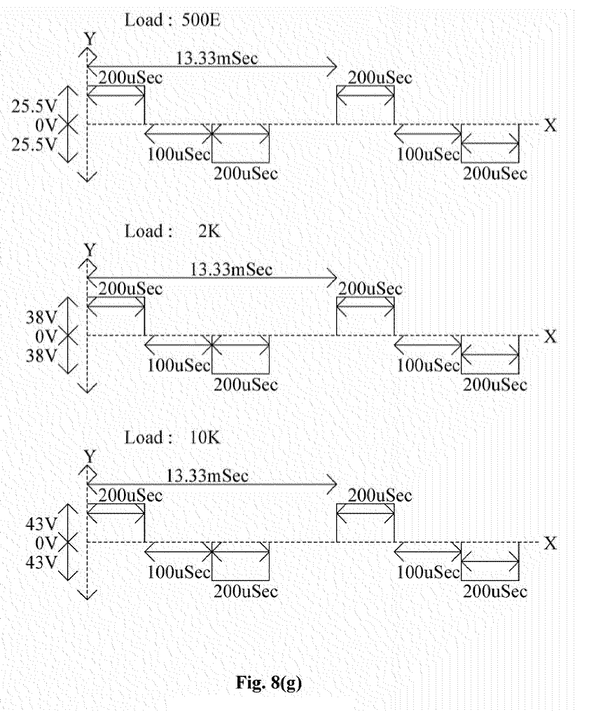

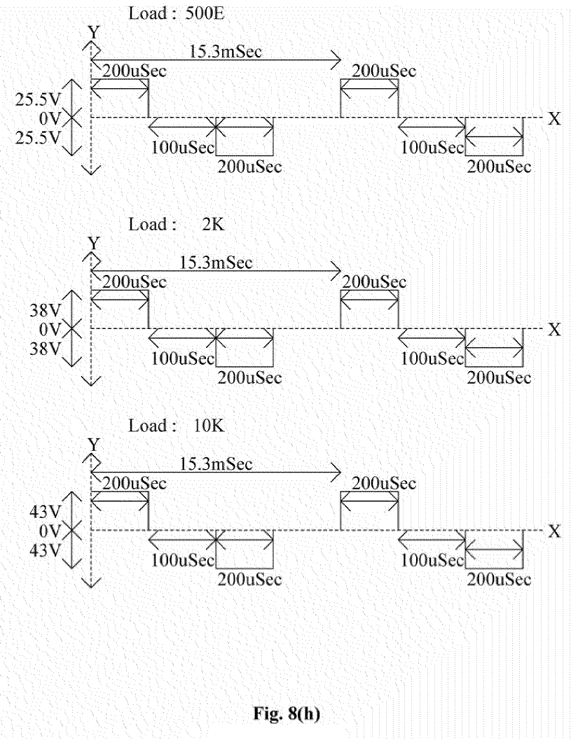

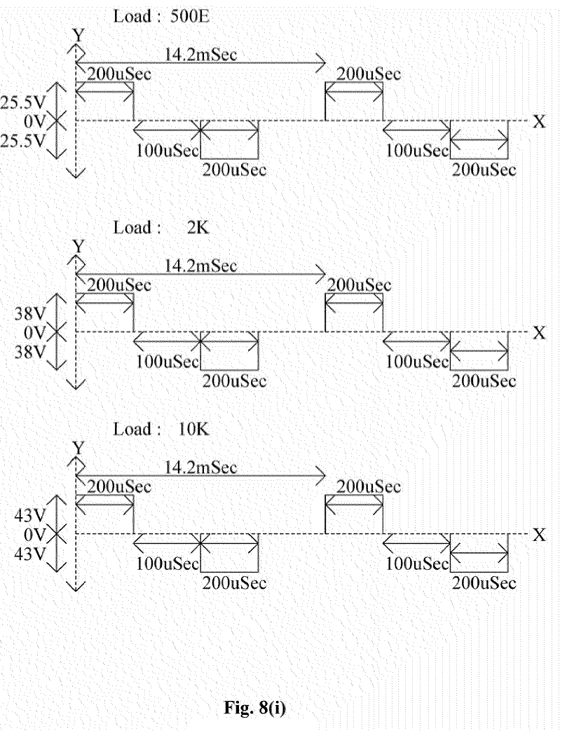

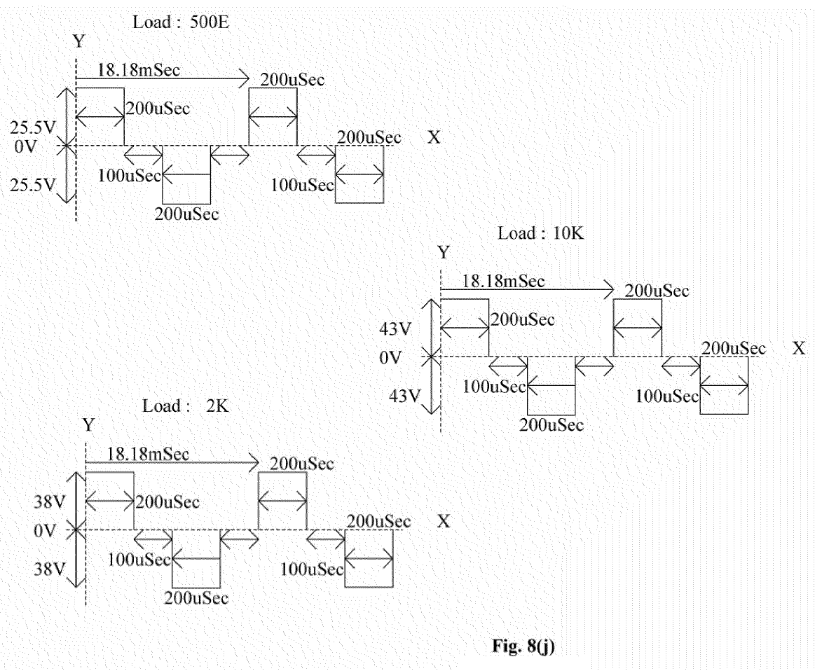

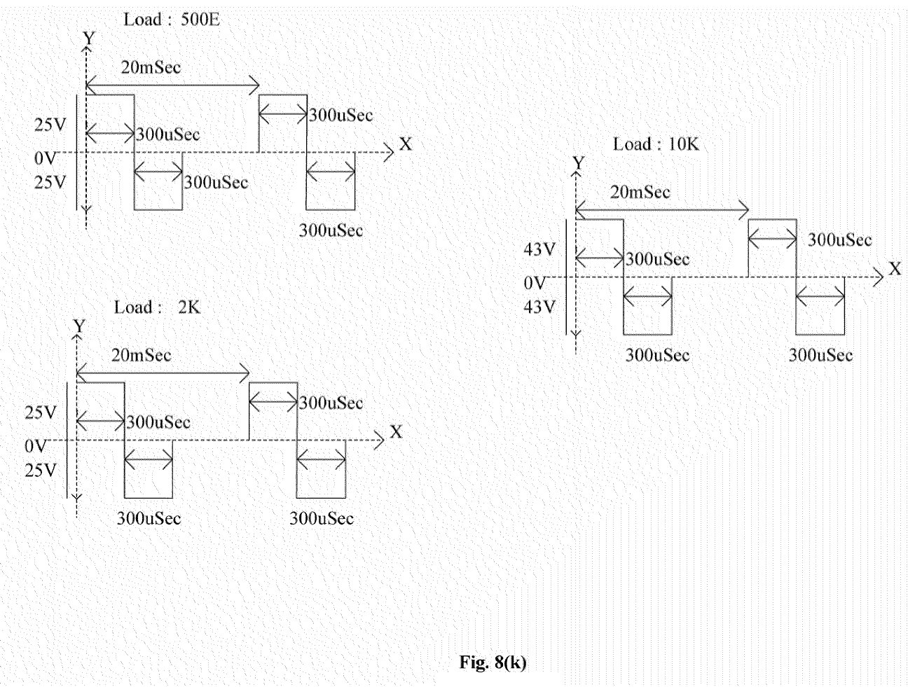

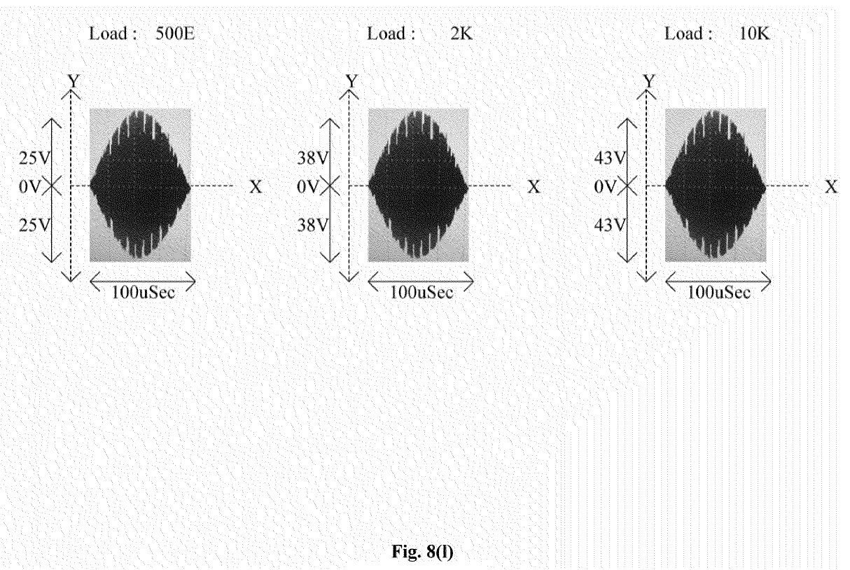

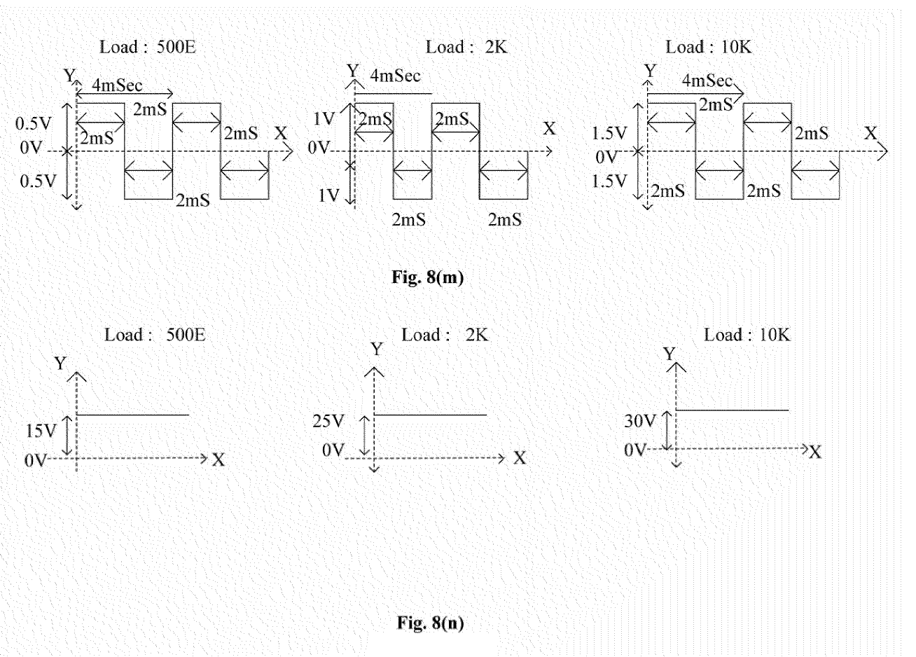

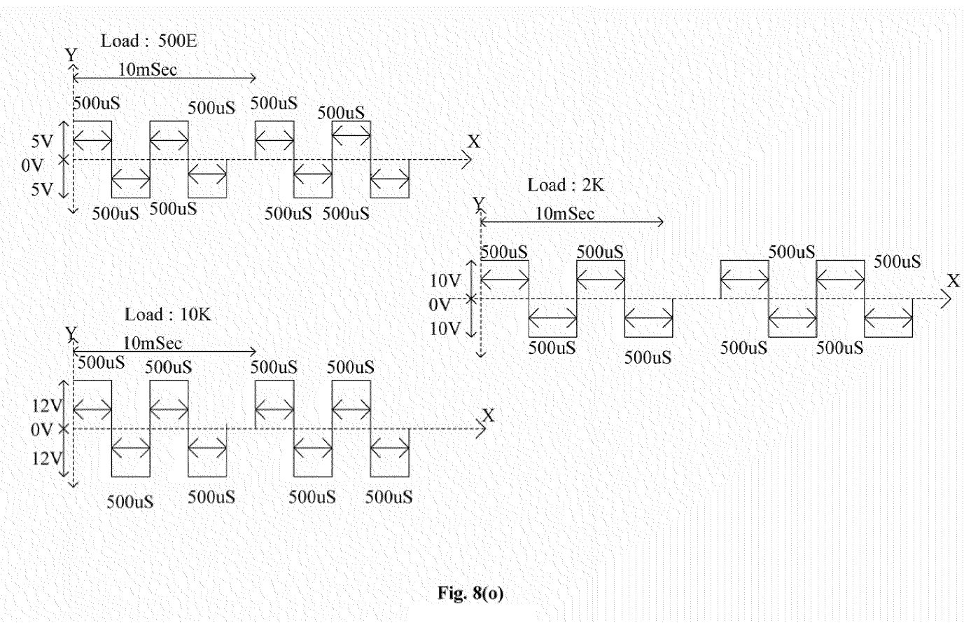

FIG. 8 (a) illustrates an output waveform with resistive loads (500 Ohms, 2000 Ohms and 10000 Ohms) using standard (4-electrode) IF. IF therapy exploits the interference of two separately generated sinusoidal currents applied to the body simultaneously as shown in FIG. 8 (a). FIG. 8 (a) illustrates IF quadric polar (4-electrode) stimulation with interference taking place within the tissue from IF therapy with an electrode placement. Interferential electrical stimulation's is a unique way of effectively delivering therapeutic frequencies to tissue. Interferential stimulators use a fixed carrier frequency of 5,000 Hz per second and also a second adjustable frequency of 5,001-5,250 Hz per second. When the fixed and adjustable frequencies combine, they produce the desired signal frequency (Interference frequency). Interferential stimulation is concentrated at the point of intersection between the electrodes. This concentration occurs deep in the tissues as well as at the surface of the skin. Thus, with Interferential Stimulators, current perfuse to greater depths and over a larger volume of tissue than other forms of electrical therapy. Treatment intensity is available up to 50 mAmp. IF stimulation can be used for the symptomatic relief and management of chronic, intractable pain.

FIG. 8 (b) to FIG. 8 (k) illustrate the output waveform for EMS with a 500 Ohms, 2000 Ohms and 10000 Ohms resistive loads. In an exemplary embodiment, the multi-function device according to the present invention, EMS stimulation (FIG. 8 (b) to FIG. 8 (k) has sequence of different pulses with different frequency and different pulse width, Treatment can be given for Abdomen, Buttocks or massage. For Abdominal stimulation (FIG. 8 (b) to FIG. 8 (f)) there are 5 sequences (Frequency: 75 Hz, 55 Hz, 70 Hz, 99 Hz and pulse width either 300 μSec or 350 μSec). Each sequence changes after 1 min of contraction and 10 sec of relaxation, and For Buttock stimulation (FIG. 8 (g) to FIG. 8 (j)) there are 4 sequences (Frequency: 75 Hz, 55 Hz, 65 Hz, and pulse width 200 μSec) Each sequence changes after 1 min of contraction and 10 sec of relaxation. EMS can be used for improvement of abdominal tone, for strengthening of the abdominal muscles, for development of firmer abdomen and strengthening, toning and firming of buttocks & thighs. In device EMS is also available for massage (FIG. 8 (k)) with 50 Hz and 300 μSec, this stimulation can be used for increase in local blood flow in the treatment area. Treatment intensity is available up to 50 mAmp.

FIG. 8 (l) illustrates exemplary Premod IF (2-electrode) output waveforms with resistive loads of 500 Ohms, 2000 Ohms and 10000 Ohms.

If therapy exploits the interference of two separately generated sinusoidal currents applied to the body, in pre modulated inferential; FIG. 8 (l) uses only two electrodes, in pre modulation Carrier (5000 Hz) and modulated frequency (5001 to 5250) is combined through software, so that the user can get pre modulated IF current. Treatment intensity is available up to 50 mAmp. Premod IF stimulation can be used for the symptomatic relief and management of chronic, intractable pain.

FIG. 8 (m) illustrates exemplary micro-current (2-electrode) output waveforms with resistive loads of 500 Ohms, 2000 Ohms and 10000 Ohms. FIG. 8 (m) is for micro current therapy It provides cosmetic benefits by tightening and lifting sagging muscle and skin. Micro-current treatment can be used to reduce or eliminate fine lines, creases and wrinkles from the skin; hydrate the skin, making it smoother and softer; improve blood circulation and lymphatic drainage; to make firm sagging skin, tighten flabby jowls, chins and cheeks; lessen the puffiness and dark circles beneath the eyes; and to reduce symptoms of sinusitis, roscoe and acne. Conducting gloves are used with the device for micro current treatment. The pulse rate varies from 0.5 Hz to 250 Hz, Pulse width is 50% of Frequency and treatment intensity is available up to 999 μAmp.

FIG. 8 (n) illustrates exemplary Galvanic-DC (2-electrode) output waveforms with resistive loads of 500 Ohms, 2000 Ohms and 10000 Ohms. Galvanic therapy can be used in acute and chronic cases involving soft tissue inflammation or muscle spasm. It helps in repairing damaged tissues, in reducing muscle spasm, in improving blood circulation. It sedates nerves and speeds the healing process. Injury to soft tissues disrupts their normal physiology resulting in atrophy, abnormal metabolism, decreased circulation and loss of function. Galvanic Therapy helps to reverse this pathological process. For Ball electrodes are with the device for galvanic stimulation. And stimulation range is up to 30 mAmp.

FIG. 8 (o) illustrates exemplary CES (Cranial electro stimulation therapy) output waveforms with resistive loads of 500 Ohms, 2000 Ohms and 10000 Ohms. CES involves the passing of small electrical impulses across the head, usually from electrodes placed on or near both ears. In the exemplary waveforms pulse rate is 100 HZ, and stimulation intensities range from 0 to 20 mA via modified square waves. CES can be used to treat a variety of chronic condition including treatment of stress, anxiety insomnia, alcoholism and drug addiction, headache, cognitive dysfunction in head injured patients, psychiatric conditions, reflex sympathetic dystrophy and multiple sclerosis.

In an exemplary embodiment, the multi-function device according to the present invention also provides pulsed Ultrasound therapy with 1 MHz and 50% duty cycle and the intensity range is up to 3 watt/cm2.Ultrasound is a therapeutic treatment method utilizing sound waves to treat pain, inflammation and muscle spasm. Ultrasound waves are applied to tissues using an Ultrasound applicator. Ultrasound gel can be used on the skin in order to reduce friction. The gel also acts as a conductor of the ultrasonic waves. The waves are generated by a piezoelectric effect caused by the vibration of crystals within the Ultrasound applicator. The ultrasonic waves that pass through the skin cause a vibration of the local tissues. This vibration or cavitations can cause a deep heating locally though usually no sensation of heat will be felt by the patient. These waves stimulate joints, ligaments, tendons, muscles and other tissues providing targeted pain relief without drugs or surgery.

In an exemplary embodiment, the multi-function device according to the present invention also provides Vacuum Therapy. Vacuum producing therapeutic massage is used for temporarily improving local blood circulation; temporary relief of minor muscle aches pains, and temporarily reducing the appearance of cellulite.

The multi-function device according to the present invention produces constant current therapy. For example, if a patient selects amplitude of 25 mA, a waveform of 25 Vpp output is generated. However, based on the patient's skin resistance, the monitored output is 25 mA up to 1.5 Kohm resistance and above 1.5 Kohm load may not be 25 mAmp. Thus, the amplitude may have to be adjusted accordingly. If the mode is a variable load mode, the device of the present invention will automatically adjust the amplitude in order to obtain the desired output.

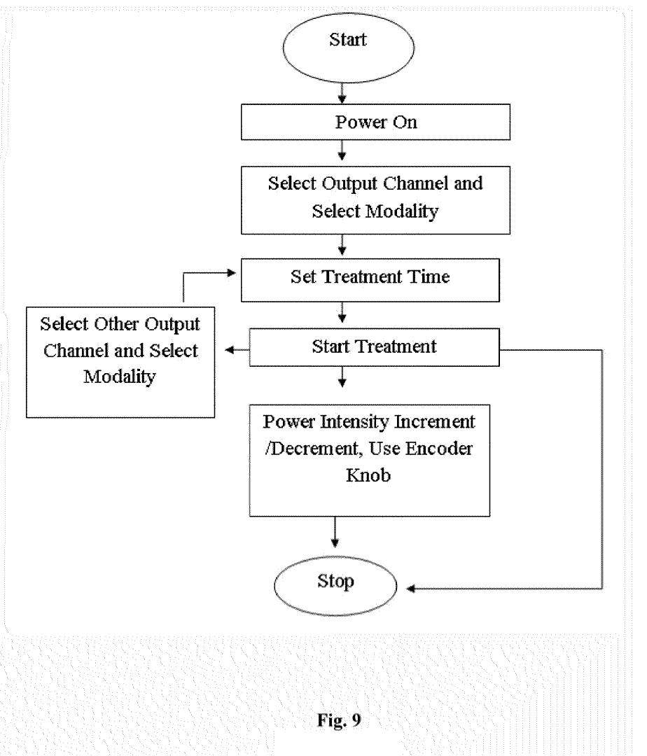

FIG. 9 is a flow diagram illustrating exemplary logic performed by the microcontroller of the present invention. The logic moves from a start block to block where power is applied. The LCD displays the modes. The modes/channels are selected by touching the respective keys. The treatment time is set. The treatment is started by touching the START button and the intensity is increased/decreased by using the encoder knob. If required, the user can also select another available mode/ channel and start another treatment simultaneously. The treatment can be stopped by touching the STOP key.

In exemplary embodiments, there is an ON/OFF button on keypads. In exemplary embodiments, when power is applied, a calibration function is performed (block 102). Once calibration has been applied, a determination of what to do is determined by obtaining parameters (block 104). Preferably, the initial display will display the first pre-defined mode with amplitude of zero. Thus, the user must either select a new mode and/or increase the amplitude in order to begin the therapy. Preferably, at any time (during any mode of operation), the user can stop the therapy (for example, by turning the machine off or setting the amplitude to zero).

As mentioned above, the device of the present invention can operate in multiple modes. In an exemplary embodiment, the multi-function device according to the present invention has the following modes:
(1) Interferential (IF) mode;
(2) EMS Mode;
(3) Pre-modulated IF mode;
(4) Micro Current mode;
(5) Galvanic-DC mode;
(6) CES Mode;
(7) Ultrasound Therapy mode; and
(8) Vacuum therapy mode;

In an exemplary embodiment, in IF stimulation maximum three outputs viz. output 1, 2, and 3 (six channels/12 electrodes) can be used. Thus, the device according to the present invention can be used to treat three body areas simultaneously with the IF stimulation (4 electrodes for each body area). In EMS stimulation two outputs viz. output 1, 2 (four channels/8 electrodes) are used. Thus, the device according to the present invention can be used to treat four body areas (abdominal/buttocks) simultaneously with the EMS stimulation. Whereas, in the massage therapy with EMS one body area is covered. In Pre-modulated IF stimulation two outputs viz. output 1, 2 (four channels/8 electrodes) are used. Thus, the device according to the present invention can be used to treat two body areas simultaneously with the Pre-modulated IF stimulation. The micro-current stimulation is given through output 4 (one channel/two conductive gloves). In Galvanic-DC mode stimulation maximum one output out of the outputs 1, 2, or 3 (two channels/4 electrodes) can be used. Thus, the device according to the present invention can be used to treat two body areas simultaneously with the Galvanic-DC stimulation (2 electrodes for each body area). The CES stimulation is given through output 4 (one channel/two ear clips). Ultrasound therapy can be given through a separate ultrasound socket with the help of an ultrasound applicator. Vacuum therapy can be given through a separate Vacuum socket with the help of vacuum cups. Thus, any of the Interferential (IF) mode, EMS Mode, Pre-modulated IF mode, Micro Current mode, Galvanic-DC mode or CES Mode can be selected concurrently depending upon the availability of the free outputs viz. 1, 2, 3 and 4. Vacuum or ultrasound therapy can be started any time irrespective of any of the other running stimulation/therapy.

Based on the foregoing, it should be appreciated that the various embodiments of the claimed subject matter may include one more other elements and certain elements may be replaced with other equivalent elements. Additional modifications and improvements of the described embodiments will also be apparent to those of ordinary skill in the art. Thus, the particular combinations of elements described and illustrated herein are intended to represent only certain embodiments of the claimed subject matter and are not intended to limit alternative combinations of devices which fall within the spirit and scope of the claimed subject matter.

What is claimed is:

1. A medical device comprising:
a. a housing;
b. at least one microprocessor for generating at least one signal,
c. at least one amplifier circuit for amplifying said one or more signals;
d. at least one feedback circuit for delivering one constant current to a patient's body,
e. at least one lead wire socket located on the exterior of said housing of the device;
f. at least one pair of electrodes connected to said one or more lead wire sockets, wherein the electrodes are placed in communication with at least one region of a patient's body for delivering an output current to said one or more skin regions of said patient's body; and
g. a controller for activating and regulating said constant current;
wherein said controller is used to activate said constant current and to select one or more selected modes for the constant current.

2. The device of claim 1, wherein the selected mode comprises:
a. an interferential mode;
b. an electric muscle stimulation mode;
c. a pre-modulated interferential mode;
d. a galvanic-DC mode;
e. a micro current mode;
f. a cranial electric stimulation mode;
g. an ultrasound therapy mode; and
h. a vacuum therapy mode.

3. The device of claim 1 further comprising a display located on the exterior of the housing of the device for providing audio and/or visual information to said user.

4. The device of claim 1, wherein four independent modes can be selected simultaneously from the following group: the ultrasound therapy mode, the vacuum therapy mode, the electric muscle stimulation mode, the pre-modulated interferential mode and the galvanic-DC mode, an interferential mode, a micro current mode and the cranial electric stimulation mode.

5. The device of claim 1, wherein the output current is up to:
a. 100 mA in each of the interferential mode, electric muscle stimulation mode and pre-modulated interferential mode;
b. 30 mA in the galvanic-DC mode;
c. 400 μA in the micro current mode;
d. 1.5 mA in cranial electric stimulation mode.

6. The device of claim 1, further comprising a microprocessor which acts both as a signal generator and a feedback signal receiver wherein said electrodes send a signal to said microprocessor containing the value of the resistance found at one or more of the contact points of the electrodes and the skin regions of the patient and wherein said microprocessor adjusts the amplification of said signal in order to maintain a constant current to said one or more electrodes.
